(19) United States
(12) Patent Application Publication
(10) Pub. No.: US 2019/0358335 A1
(43) Pub. Date: Nov. 28, 2019

(54) STOMACH ACID-STABLE AND MUCIN-BINDING PROTEIN-POLYMER CONJUGATES

(71) Applicant: Alan J. RUSSELL, Gibsonia, PA (US)

(72) Inventors: Alan J. Russell, Gibsonia, PA (US); Stefanie Baker, Pittsburgh, PA (US)

(21) Appl. No.: 16/477,843

(22) PCT Filed: Jan. 12, 2018
(86) PCT No.: PCT/US2018/013552

§ 371 Date: Jul. 12, 2019

Related U.S. Application Data
(60) Provisional application No. 62/498,988, filed on Jan. 12, 2017.

(51) Int. Cl. A61K 47/58; A61K 38/48

(52) U.S. Cl. CPC A61K 47/58; A61K 38/4826

(57) ABSTRACT

Provided herein are protein-polymer conjugates, pharmaceutical compositions including protein-polymer conjugates, and methods of using the same, e.g., in therapeutic and industrial applications.

### CLAIM OF PRIORITY

This application claims the benefit of U.S. Provisional Application Ser. No. 62/498,988, filed on Jan. 12, 2017. The entire contents of the foregoing are incorporated herein by reference.

### TECHNICAL FIELD

The following disclosure relates to protein-polymer conjugates and methods of using the same, e.g., in therapeutic and industrial applications.

### BACKGROUND

Proteins are used in a multitude of industrial and therapeutic applications. For example, therapeutic proteins have been approved for the treatment of a variety of diseases and conditions such as inflammatory and gastrointestinal diseases. However, because proteins are susceptible acid-induced unfolding, their use is limited to environments having a pH range that supports tertiary structure stability of the protein. For instance, it is highly desireable to administer some therapeutic proteins; however, stomach acid and proteases rapidly leads to the unfolding and degradation of proteins prior to absorption. Therefore, many proteins are administered via non-oral routes in order to avoid the upper digestive tract. There is a need for strategies that stabilize proteins in acidic environments while maintaining their functionality.

### SUMMARY

In one aspect, provided herein is a protein-polymer conjugate, comprising at least one polymer covalently conjugated to a protein, wherein the at least one polymer stabilizes a partially unfolded state of the conjugated protein when the conjugate is in the environment having a pH of about 3.0 or less, and wherein the conjugate is resistant to complete denaturation in the environment. In some embodiments, the conjugate is resistant to complete denaturation in an environment having a pH of about 1.0. In some embodiments, the at least one polymer comprises from about 10 monomeric units to about 200 monomeric units.

In some embodiments, the conjugated protein is capable of refolding to a native state when the conjugate is subsequently in an environment having a pH above about 3.0.

In some embodiments, the conjugated protein is capable of refolding to a native state when the conjugate is subsequently in an environment having a pH of from about 5.5 to about 8.5.

In some embodiments, the protein is selected from the group consisting of an antibody, an Fc fusion protein, an enzyme, an anti-coagulation protein, a blood factor, a bone morphogenetic protein, a growth factor, an interferon, an interleukin, a thrombolytic agent, a protein or peptide antigen, and a hormone. In some embodiments, the protein is an enzyme selected from the group consisting of lactase, xylanase, chymotrypsin, trypsin, and a gluten-degrading enzyme. In some embodiments, the enzyme is chymotrypsin. In some embodiments, the protein is selected from the group consisting of insulin, oxytocin, vasopressin, adrenocorticotrophic hormone, prolactin, luliberin, growth hormone, growth hormone releasing factor, parathyroid hormone, somatostatin, glucagon, interferon, gastrin, tetragastrin, pentagastrin, urogastroine, secretin, prosecretin, calcitonin, angiotensin, renin, glucagon-like peptide-1, and human granulocyte colony stimulating factor.

In some embodiments, when the conjugate is in an environment having a pH of about 3.0 or less, the conjugated protein has a half-life of at least about 125% of the half-life of the protein in its native state when exposed to an environment having a pH of about 3.0 or less.

In some embodiments, the conjugated protein is an enzyme, and the enzyme retains at least about 50% of its enzymatic activity when the conjugate is in an environment having a pH of 3.0 or less. In some embodiments, the conjugated protein is an enzyme, and the enzyme retains at least about 75% of its enzymatic activity when the conjugate is in an environment having a pH of 3.0 or less. In some embodiments, the conjugated protein is an enzyme, and the enzyme retains at least about 85% of its enzymatic activity when the conjugate is in an environment having a pH of 3.0 or less. In some embodiments, the environment has a pH of about 1.0.

In some embodiments, the conjugate is made by growing at least one polymer directly from the surface of the protein using atom-transfer radical polymerization (ATRP).

In some embodiments, the conjugate comprises a plurality of polymers. In some embodiments, the plurality of polymers comprises at least 4 polymers. In some embodiments, the plurality of polymers is made by growing the polymers directly from the surface of the protein using atom-transfer radical polymerization (ATRP). In some embodiments, each polymer in the plurality of polymers comprises monomeric units of the same type. In some embodiments, the plurality of polymers comprises a first polymer and a second polymer, wherein the first polymer and the second polymer are each comprised of monomeric units of a different type.

In some embodiments, the at least one polymer comprises a positively charged polymer, a zwitterionic polymer, or a combination thereof. In some embodiments, the positively-charged polymer is poly(quaternary ammonium methacrylate) (pQA). In some embodiments, the zwitterionic polymer is poly(carboxybetaine acrylamide) (pCBAm), poly(carboxybetaine methacrylate) (pCBMA), or poly(sulfobetaine methacrylamide) (pSBAm).

In some embodiments, the conjugate is specifically binds to mucin.

In some embodiments, the protein is chymotrypsin and the at least one polymer is pQA. In some embodiments, the protein is chymotrypsin and the at least one polymer is pCBAm.

In another aspect, provided herein is a mucoadhesive protein-polymer conjugate, comprising at least one polymer covalently conjugated to a protein, wherein the conjugate is capable of binding to mucin.

In some embodiments, the conjugate is made by growing at least one polymer directly from the surface of the protein using atom-transfer radical polymerization (ATRP).

In some embodiments, the at least one polymer is a positively-charged polymer or a zwitterionic polymer. In some embodiments, the positively-charged polymer is poly(quaternary ammonium methacrylate) (pQA). In some embodiments, the zwitterionic polymer is poly(carboxybetaine acrylamide) (pCBAm), poly(carboxybetaine methacrylate) (pCBMA), or poly(sulfobetaine methacrylamide) (pSBAm). In some embodiments, the zwitterionic polymer is pCBAm.

In some embodiments, the conjugated protein does not bind to mucin in its native state.

In some embodiments, the conjugated protein is selected from the group consisting of an antibody, an Fc fusion protein, an enzyme, an anti-coagulation protein, a blood factor, a bone morphogenetic protein, a growth factor, an interferon, an interleukin, a thrombolytic agent, a protein or peptide antigen, and a hormone. In some embodiments, the conjugated protein in an enzyme, and the enzyme is selected from the group consisting of lactase, xylanase, chymotrypsin, trypsin, a gluten-degrading enzyme. In some embodiments, the conjugated protein is chymotrypsin. In some embodiments, the conjugated protein is selected from the group consisting of insulin, oxytocin, vasopressin, adrenocorticotrophic hormone, prolactin, luliberin, growth hormone, growth hormone releasing factor, parathyroid hormone, somatostatin, glucagon, interferon, gastrin, tetragastrin, pentagastrin, urogastroine, secretin, prosecretin, calcitonin, angiotensin, renin, glucagon-like peptide-1, and human granulocyte colony stimulating factor.

In some embodiments, the at least one polymer comprises from about 10 monomeric units to about 200 monomeric units.

In some embodiments, the conjugated protein is chymotrypsin and the at least one polymer is pQA. In some embodiments, the conjugated protein is chymotrypsin and the at least one polymer is pCBAm.

In some embodiments, the conjugate comprises a plurality of polymers. In some embodiments, the plurality of polymers comprises at least 4 polymers. In some embodiments, the plurality of polymers is made by growing the polymers directly from the surface of the protein using atom-transfer radical polymerization (ATRP). In some embodiments, each polymer in the plurality of polymers comprises monomeric units of the same type. In some embodiments, the plurality of polymers comprises a first polymer and a second polymer, wherein the first polymer and the second polymer are each comprised of monomeric units of a different type.

In another aspect, provided herein is a composition comprising a protein-polymer conjugate described herein. In some embodiments, the composition is a pharmaceutical dosage form comprising a pharmaceutically acceptable excipient. In some embodiments, the composition is formulated for oral, rectal, intranasal, or intravaginal administration to a subject. In some embodiments, the composition is a foodstuff.

In another aspect, provided herein is a method of enhancing the delivery of a protein to the intestinal tract of a subject, the method comprising administering to the subject a pharmaceutical composition comprising a protein-polymer conjugate, wherein the conjugate comprises at least one polymer covalently conjugated to a protein, wherein the at least one polymer stabilizes a partially unfolded state of the conjugated protein when the conjugate is in the environment having a pH of about 3.0 or less, and wherein the conjugate is resistant to complete denaturation in the environment. In some embodiments, the conjugate is resistant to complete denaturation when exposed to an environment having a pH of about 1.0.

In some embodiments, when the conjugate is in an environment having a pH of about 3.0 or less, the conjugated protein has a half-life of at least about 125% of the half-life of the protein in its native state when exposed to an environment having a pH of about 3.0 or less. In some embodiments, the conjugated protein is an enzyme, and the enzyme retains at least about 50% of the enzymatic activity of the native enzyme when the conjugate is in an environment having a pH of about 3.0 or less. In some embodiments, the conjugated protein is an enzyme, and the enzyme retains at least 75% of the enzymatic activity of the native enzyme when the conjugate is in an environment having a pH of about 3.0 or less. In some embodiments, the conjugated protein is an enzyme, and the enzyme retains at least about 85% of the enzymatic activity of the native enzyme when the conjugate is in an environment having a pH of about 3.0 or less. In some embodiments, the environment has a pH of about 1.0. In some embodiments, the environment having a pH of about 3.0 or less is the stomach of the subject.

In some embodiments, the conjugated protein refolds to a native state when the conjugate is in an environment having a pH of from about 5.5 to about 8.5. In some embodiments, the environment having a pH of from about 5.5 to about 8.5 is the small intestine, the large intestine, or a portion thereof, of the subject.

In some embodiments, the at least one polymer comprises a positively-charged polymer, a zwitterionic polymer, or a combination thereof. In some embodiments, the positively-charged polymer is poly(quaternary ammonium methacrylate) (pQA). In some embodiments, the zwitterionic polymer is poly(carboxybetaine acrylamide) (pCBAm), poly(carboxybetaine methacrylate) (pCBMA), or poly(sulfobetaine methacrylamide) (pSBAm).

In some embodiments, the conjugate is capable of binding to mucin.

In another aspect, provided herein is a method of targeting the delivery of a protein to the gastrointestinal tract of a subject, the method comprising administering to the subject a pharmaceutical composition comprising a mucoadhesive protein-polymer conjugate and a pharmaceutically acceptable excipient, wherein the conjugate comprises at least one polymer covalently conjugated to a protein, wherein the at least one polymer stabilizes a partially unfolded state of the conjugated protein when the conjugate is in the environment having a pH of about 3.0 or less, and wherein the conjugate is resistant to complete denaturation in the environment. In some embodiments, the protein does not bind to mucin in its native state.

In some embodiments, the at least one polymer is a positively-charged polymer or a zwitterionic polymer. In some embodiments, the positively-charged polymer is poly(quaternary ammonium methacrylate) (pQA). In some embodiments, the zwitterionic polymer is poly(carboxybetaine acrylamide) (pCBAm), poly(carboxybetaine methacrylate) (pCBMA), or poly(sulfobetaine methacrylamide) (pSBAm). In some embodiments, the zwitterionic polymer is pCBAm.

In some embodiments, the protein is selected from the group consisting of an antibody, an Fc fusion protein, an enzyme, an anti-coagulation protein, a blood factor, a bone morphogenetic protein, a growth factor, an interferon, an interleukin, a thrombolytic agent, a protein or peptide antigen, and a hormone. In some embodiments, the protein is an enzyme selected from the group consisting of lactase, xylanase, chymotrypsin, trypsin, a gluten-degrading enzyme. In some embodiments, the enzyme is chymotrypsin. In some embodiments, the protein is selected from the group consisting of insulin, oxytocin, vasopressin, adrenocorticotrophic hormone, prolactin, luliberin, growth hormone, growth hormone releasing factor, parathyroid hormone, somatostatin, glucagon, interferon, gastrin, tetragastrin, pentagastrin, urogastroine, secretin, prosecretin, calcitonin, angiotensin, renin, glucagon-like peptide-1, and human granulocyte colony stimulating factor.

In some embodiments, the at least one polymer comprises from about 10 monomeric units to about 200 monomeric units.

In some embodiments, the conjugate is made by growing the at least one polymer directly from the surface of the protein using atom-transfer radical polymerization (ATRP).

In some embodiments, the protein is chymotrypsin and the at least one polymer is pQA. In some embodiments, the protein is chymotrypsin and the at least one polymer is pCBAm.

In some embodiments, the conjugate comprises a plurality of polymers. In some embodiments, the plurality of polymers comprises at least 4 polymers. In some embodiments, the plurality of polymers is made by growing the polymers directly from the surface of the protein using atom-transfer radical polymerization (ATRP). In some embodiments, each polymer in the plurality of polymers comprises monomeric units of the same type. In some embodiments, the plurality of polymers comprises a first polymer and a second polymer, wherein the first polymer and the second polymer are each comprised of monomeric units of a different type.

In some embodiments, the pharmaceutical composition is administered to the subject orally, intraocularly, intranasally, intravaginally, or rectally.

In some embodiments, the conjugated protein exhibits reduced immunogenicity as compared to the protein in its native state.

In some embodiments, the subject has a disease or disorder selected from the group consisting of autism, cystic fibrosis, and exocrine pancreatic insufficiency.

One aspect features polymers which can stabilize any protein, and the method of doing so by controlling and preventing the interaction of the polymer and the protein surface. Another aspect features protein-polymer conjugates which bind to mucin. In another aspect, polymer-based protein engineering is used to synthesize different chymotrypsin-polymer conjugates. In some examples, this is done using “grafting-from” atom transfer radical polymerization. In another aspect, polymer charge can be used to influence chymotrypsin-polymer conjugate mucin binding, bioactivity, and stability in stomach acid. One aspect features cationic polymers covalently attached to chymotrypsin, which showed high mucin binding. Another aspect features stabilized enzyme hybrids.

Certain implementations may provide one or more advantages. For example, the mucoadhesive protein-polymer conjugates provided herein have increased residence time in the intestinal tract or when associated with any biological tissue that contains accessible mucin as compared to the unconjugated protein. In addition or as an alternative, in certain implementations, when polymers are covalently attached to the surface of a protein, the degree to which those polymers interact with the protein surface is the predominant determinant of whether the polymer will stabilize or inactivate the protein; preferential interactions between the polymer and the protein lead to removal of water from the surface of the protein and this inactivates the enzyme. Also, in certain implementations, cationic polymers also increased chymotrypsin activity from pH 6-8 and decreased the tendency of chymotrypsin to structurally unfold at extremely low pH. Further, the reduced immunogenicity and increased stability of certain implementations of the protein-polymer conjugates described herein makes their use in therapeutic applications particularly attractive.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 depicts schematic representations of CT-pOEGMA, CT-pCBAm (+/−), CT-pSMA (−), and CT-pQA (+). The charge state of each polymer is shown at pH 7; below pH 4.5 the carboxylic acid in pCBAm was protonated and pCBAm had an overall positive charge. The charge states of the other polymers have no pH-dependence from pH 1-8.

FIGS. 2A-2D show the dependence of chymotrypsin-polymer hydrodynamic diameter on charge state of the polymer. CT-pCBAm (+/−) (26.3±3.2 nm; FIG. 2A), CT-pOEGMA (20.1±2.0 nm; FIG. 2B), CT-pQA(+) (34.5±2.9 nm; FIG. 2C), and CT-pSMA (−) (17.2±2.2 nm; FIG. 2D) hydrodynamic diameter values were measured by dynamic light scattering (DLS) in 50 mM sodium phosphate (pH 7.0, 25° C.). Native chymotrypsin hydrodynamic diameter is considerably smaller than that of the conjugates (5.7±2 nm).

FIGS. 3A-3F show the pH-Dependence of mucin-particle crosslinking by ATRP-synthesized free polymers. FIG. 3A: pH 1.0 (167 mM HCl); FIG. 3B: pH 4.5 (50 mM ammonium acetate buffer); FIG. 3C: pH 8 (50 mM sodium phosphate buffer); FIG. 3D: 167 mM HCl with 10% ethanol or 0.2 M NaCl; FIG. 3E: 50 mM ammonium acetate with ethanol or NaCl; and FIG. 3F: 50 mM sodium phosphate with 10% ethanol, 0.2 M NaCl, or 0.5 M NaCl. Normalized absorbance at 400 nm (turbidity) at 37° C. was used as a marker for mucin-particle crosslinking by free polymer.

FIGS. 4A-4C show the pH dependence of mucin particle crosslinking by chymotrypsin-polymer conjugates. FIG. 4A: pH 1.0 (167 mM HCl); FIG. 4B: pH 4.5 (50 mM ammonium acetate buffer); and FIG. 4C: pH 8 (50 mM sodium phosphate buffer). Chymotrypsin polymer conjugates exhibited mucin binding properties consistent with free polymers. At pH 1.0 and pH 4.5, only enzyme conjugates structurally stable to those conditions (CT-pQA, CT-pCBAm) were tested to eliminate the effect of unfolded protein. Native protein showed no mucin-binding properties at equivalent concentrations.

FIGS. 5A-5F show the pH dependence of kinetics for chymotrypsin- and chymotrypsin-polymer conjugate-catalyzed hydrolysis of a negatively charged substrate. Kinetic constants k_{cat} (FIG. 5A), K_{M} (FIG. 5B), and k_{cat}/K_{M} (FIG. 5C) were measured for native chymotrypsin (open upside down triangle) from pH 6-8 at 37° C. in 100 mM sodium phosphate buffer. Relative kinetic constants k_{cat} (FIG. 5D), K_{M} (FIG. 5E), and k_{cat}/K_{M} (FIG. 5F) were calculated for CT-pSMA (diamond), CT-pOEGMA (triangle), CT-pQA (circle), and CT-pCBAm (square) in the same conditions and plotted relative to native chymotrypsin.

FIGS. 6A and 6B depict the rate of acid-mediated irreversible inactivation of chymotrypsin-polymer conjugates. FIG. 6A: native chymotrypsin (upside down triangle), CT-pCBAm (square), CT-pOEGMA (triangle), CT-pQA (+) (circle), CT-pSMA (−) (diamond) were incubated in 167 mM HCl at 37° C. FIG. 6B: native CT (upside down triangle) was incubated with pOEGMA (triangle), pCBAm (square), pSMA (−) (diamond), and pQA (+) (circle) free polymers. Activity assays were completed using 288 μM substrate (NS-AAPF-pNA) in 100 mM sodium phosphate (pH 8.0) at 37° C.

FIG. 7 depicts acid-mediated changes in chymotrypsin-polymer conjugate tertiary structure. Tryptophan intrinsic fluorescence wavelength of maximum emission intensity values (λₘₐₓ) after incubation in 167 mM HCl (pH 1) at 37° C. for native chymotrypsin (upside down triangle) and chymotrypsin conjugates; CT-pCBAm (square), CT-pOEGMA (triangle), CT-pQA (+) (circle), CT-pSMA (−) (diamond). An increase in λₘₐₓ indicates protein unfolding. Time=0 minutes indicates tryptophan intrinsic fluorescence at pH 8, 37° C.

FIGS. 8A and 8B show the electrostatic potential coulombic surface coloring for CT-Br. CT-Br structures were obtained after a 10 ns molecular dynamics simulations in water. Molecular graphics and surface charge analyses were performed with the UCSF Chimera package at neutral pH 7.0 (FIG. 8A) and pH 1.0 (FIG. 8B). The PROPKA method was used for the prediction of the ionization states in the initiator complex at both pH values.

FIG. 9 depicts the hypothesized effect of polymer conjugation on hydration shell of chymotrypsin. For CT-pSMA(−) and CT-pOEGMA, the polymers interacted with chymotrypsin, displacing water molecules via preferential binding which resulted in a decrease in stability. Conversely, CT-pQA (+) and CT-pCBAm(+) were excluded from chymotrypsin due to unfavorable interactions between polymer and protein, resulting in preferential hydration which increased stability to strongly acidic conditions.

FIGS. 10A-10C shows the synthesis and characterization of chymotrypsin-polymer conjugates. FIG. 10A shows an exemplary synthesis scheme to prepare “grafted-from” conjugates. The first step is initiator immobilization using surface accessible primary amines followed by atom-transfer radical polymerization (ATRP) from the initiator modified sites. FIG. 10B shows polymers of varying charge and hydrophobicity used to create conjugates using ATRP. Three conjugates with increasing chain length were created for each monomer type. FIG. 10C shows the conjugate characterization using bichinchoninic acid (BCA) assay for protein content, estimated degree of polymerization (DP) from BCA, cleaved polymer molecular weight and dispersity from gel permeation chromatography, number intensity hydrodynamic diameter (Dₕ), and zeta potential. Conjugates increased in DP for each monomer type with a corresponding increase in molecular weight and Dh. Conjugate characterization was compared to native CT and initiator modified CT (CTBr).

FIGS. 11A-11F show the Michaelis-Menten kinetics of CT conjugates at pH 4, 6, 8, and 10 (x-axis) in comparison to native CT for turnover rate (k_{cat}, s⁻¹, 1^{st} column), Michaelis constant (K_{M}, μM, 2^{nd} column), and overall catalytic efficiency (k_{cat}/K_{M}, μM⁻¹ s⁻¹, 3^{rd} column). FIG. 11A shows the kinetics of native CT (circles). FIG. 11B shows the kinetics of CT-pCBMA (±) normalized to native CT. FIG. 11C shows the kinetics of CT-pOEGMA (0) normalized to native CT. FIG. 11D shows the kinetics of CT-pDMAEMA (+/0) normalized to native CT. FIG. 11E shows the kinetics of CT-pQA (+) normalized to native CT. FIG. 11F shows the kinetics of CT-pSMA (−) normalized to native CT. Normalized native CT (dashed line), short length conjugates (diamonds), medium length conjugates (squares), and long length conjugates (triangles). Changes in activity derived from changes in K_{M} due to polymer charge and were not length dependent. Error bars represent the standard deviation from triplicate measurements.

FIGS. 12A-12G show the conjugate acid stability at pH 1 (167 mM HCl) in comparison to native CT (circles in all plots) and CTBr in terms of residual activity over 60 min and tryptophan fluorescence intensity percent change from refolding at pH 8 after 40 minutes incubation at pH 1. Residual activity for native CT (circles) and CTBr (triangles) (FIG. 12A); CT-pCBMA (±) (FIG. 12B); CT-pOEGMA (0) (FIG. 12C); CT-pDMAEMA (+/0) (FIG. 12D); CT-pQA (+) (FIG. 12E); and CT-pSMA (−) (FIG. 12F) are depicted. Native CT (circles), short length conjugates (diamonds), medium length conjugates (squares), and long length conjugates (triangles). FIG. 12G is a table depicting the tryptophan fluorescence (FL) intensity (em.350 nm/em.330 nm) percent change from 40 minutes at pH 1 to its time 0 (pH 8) indicating ability to refold for all conjugates. Top line in each column represents short length conjugates, Middle line in each column represents medium length conjugates, and bottom line in each column represents long length conjugates. Long, hydrophilic polymers, pCBMA and pQA, stabilized conjugates the most at pH 1 and were able to refold the greatest (corresponding to the lowest FL % change). All conjugates followed a one-phase decay where native CT followed a two-phase decay. Error bars represent the standard error of the mean from triplicate measurements.

FIGS. 13A-13G show the tertiary structure changes of conjugate acid stability at pH 1 (167 mM HCl) in comparison to native CT (circles in all plots) and CTBr in terms of tryptophan fluorescence (FL) intensity over time at pH 1. Tryptophan FL for native CT (circles) and CTBr (triangles) (FIG. 13A); CT-pCBMA (±) (FIG. 13B); CT-pOEGMA (0) (FIG. 13C); CT-pDMAEMA (+/0) (FIG. 13D); CT-pQA (+) (FIG. 13E); and CT-pSMA (−) (FIG. 13F) are depicted. Native CT (circles), short length conjugates (diamonds), medium length conjugates (squares), and long length conjugates (triangles). All conjugates unfold to relatively the same degree independent of length or charge and all unfolding occurs within the first 5 minutes. FIG. 13G depicts the unfolding pathways for native CT and CT-conjugates at pH 1. Polymers stabilize partially unfolded states and prevent irreversible denaturation. The ability to reversibly refold depends on polymer hydrophobicity and length. Long, hydrophilic polymers, pCBMA (±) and pQA (+), increase refolding rates by minimizing interactions with the exposed protein core. Error bars represent the standard error of the mean from triplicate measurements.

FIGS. 14A-14G show the conjugate base stability at pH 12 (10 mM NaOH) in comparison to native CT (circles in all plots) and CTBr in terms of residual activity over 60 min and tryptophan fluorescence intensity percent change from refolding at pH 8 after 40 minutes incubation at pH 12. Residual activity for native CT (circles) and CTBr (open triangles) (FIG. 14A); CT-pCBMA (±) (FIG. 14B); CT-pOEGMA (0) (FIG. 14C) CT-pDMAEMA (+/0) (FIG. 14D); CT-pQA (+) (FIG. 14E); and CT-pSMA (−) (FIG. 14F) are depicted. Native CT (circles), short length conjugates (diamonds), medium length conjugates (squares), and long length conjugates (triangles). FIG. 14G is a table depicting the tryptophan fluorescence (FL) intensity (em.350 nm/em.330 nm) percent change from 40 minutes at pH 12 to its time 0 (pH 8) indicating ability to refold for all conjugates. Top line in each column represents short length conjugates, middle line in each column represents medium length conjugates, and bottom line in each column represents long length conjugates. Conjugated polymers did not stabilize CT for any charge or chain length. All conjugates followed a two-phase decay similar to native CT. Error bars represent the standard error of the mean from triplicate measurements.

FIGS. 15A-15G show the tertiary structure changes of conjugate base stability at pH 12 (10 mM NaOH) in comparison to native CT (circles in all plots) and CTBr in terms of tryptophan fluorescence (FL) intensity over time at pH 12. Tryptophan FL for native CT (circles) and CTBr (open triangles) (FIG. 15A); CT-pCBMA (±) (FIG. 15B); CT-pOEGMA (0) (FIG. 15C); CT-pDMAEMA (+/0) (FIG. 15D); CT-pQA (+) (FIG. 15E); and CT-pSMA (−) (FIG. 15F) are depicted. Native CT (circles), short length conjugates (diamonds), medium length conjugates (squares), and long length conjugates (triangles). All conjugates unfold slowly over time independent of polymer type. FIG. 15G shows the unfolding pathways for native CT and CT-conjugates at pH 12. Conjugated polymers do not stabilize partially unfolded states and irreversible denaturation proceeds, most likely do due deprotonation of exposed tyrosine residues (pKₐ=10.5) and loss of secondary structure. Error bars represent the standard error of the mean from triplicate measurements.

FIG. 16 shows the monomer hydrophobicity as the distribution coefficient between octanol and water (log D) determined using ChemAxon at pH 1 (*), 7 (#), and 12 (\hat{ }). Hydrophobicity increases at pH 7 from QA<CBMA<SMA<DMAEMA<OEGMA.

FIG. 17 shows the matrix assisted laser desorption/ionization time-of-flight mass spectroscopy (MALDI-ToF MS) of native CT (black) and initiator modified CT (CTBr, gray). The difference in m/z allows calculation of how many modification sites were achieved. CT was modified with 12 initiators for atom-transfer radical polymerization.

FIG. 18 is a table depicting the atom-transfer radical polymerization conditions for conjugate synthesis. Reactions were performed at 4° C. to prevent CT autolysis. Increasing chain length was achieved by increasing the initiator to monomer ratio ([I]:[M]).

FIGS. 19A-19E show the residual activity measurements for stability of short length CT-conjugates at pH 1 while independently doping in 1.0 M NaCl or 10 v/v % dimethyl sulfoxide (DMSO) to disrupt electrostatic and hydrophobic interactions, respectively. In all plots, native CT (dashed line), native CT with NaCl (dashed line, circles), and native CT with DMSO (dashed line, triangles). CT-polymer (dotted line), CT-polymer with 1.0 M NaCl (dotted line, circles), CT-polymer with 10 v/v % DMSO (dotted line, triangles). CT-pCBMA (FIG. 19A); CT-pOEGMA (FIG. 19B); CT-pQA (FIG. 19C); CT-pSMA (FIG. 19D); and CT-pDMAEMA (FIG. 19E). The addition of NaCl and DMSO did not increase stability indicating an alternative mechanism for conjugate stabilization. Error bars represent standard error of the mean from triplicate measurements.

FIG. 20 is a table showing the kinetic rates of residual activity measurements for conjugates at pH 1 and pH 12. Conjugates follow a one-phase decay at pH 1 and a two-phase decay at pH 12 indicating different unfolding pathways. Rates were calculated using one-phase and two-phase decay fitting in GraphPad. Values shown are the mean with standard error from triplicate measurements.

### DETAILED DESCRIPTION

Provided herein are protein-polymer conjugates that can be used to stabilize and/or protect a protein from denaturation in an acidic environment. The protein-polymer conjugates can be used in a variety of applications including medical and industrial applications where it is desireable to stabilize a protein in acidic environments. For example, the protein-polymer conjugates described herein may be used to prevent the denaturation of a therapeutic protein from the acidic environment of the stomach during oral administration in order to improve the half-life, efficacy, and/or activity of the therapeutic protein.

The compositions and methods described herein are based on the surprising discovery that protein-polymer conjugates can be used to protect and/or stabilize a protein from denaturation at a pH lower than 3.0. This discovery can be used to stabilize any protein of interest, but is particularly useful in the development of pharmaceuticals for oral administration and industrial applications where methods are performed under acidic conditions. For example, for oral enzyme replacement therapy to be optimally effective in the GI tract, it is desireable that the enzyme remain stable from pH 1 to 8. Using the methods described herein, enzymes used for enzyme replacement therapy can be stabilized to increase their stability for oral delivery to a subject. This may, in some implementations, permit the use of lower dosages of the therapeutic.

Also provided herein are mucoadhesive protein-polymer conjugates that may be used to target a therapeutic protein to the mucosa of the gastrointestinal tract of a subject. The mucosal innermost lining of the GI tract is replete with the glycosylated protein mucin, which is known to bind charged and hydrophilic polymers. One approach to target therapeutic protein to the intestinal tract is to combine the protein with a mucoadhesive molecules (see, e.g., Smart Adv. Drug Delivery Rev. 57, 1556 (2005); and Davidovich-Pinhas and Bianco-Peled Expert Opin. Drug Delivery 7, 259 (2010)). Conjugation of a polymer to a protein of interest (e.g., a therapeutic protein) as described herein can advantageously be used to target any protein, but particularly proteins that do not have the ability to bind to mucin, the ability to do so. The conjugates may be used to target the conjugate to any mucin-containing tissue site, thereby allowing the protein to be absorbed at, or perform an activity, at said site. In some embodiments, protein-polymer conjugates have increased residence time in the intestinal tract or when associated with any biological tissue that contains accessible mucin.

Polymer-protein conjugates have become central components of the biologic drug, synthetic and food industries. Although any protein can be conjugated to a polymer as described herein, the Examples herein use the exemplary enzyme, chymotrypsin. Chymotrypsin has become one of the most commonly studied protein-polymer conjugates because of the wealth of published information about the amino acid sequence, crystal structure, and substrate preferences under a host of reaction conditions (see, e.g., Hong et al. J. Mol. Catal. B: Enzym. 42, 99 (2006); Falatach et al. Polymer 72, 382 (2015); Sandanaraj et al. J. Am. Chem. Soc. 127, 10693 (2005); Blow Biochem. J., 112, 261 (1969); Scheidig et al. Protein Sci. 6, 1806 (1997); Günther et al. Eur. J. Biochem. 267, 3496 (2000); and Wysocka et al. Protein Pept. Lett. 15, 260 (2008)). Chymotrypsin is stable over a reasonably wide pH range and in many organic solvents (see, e.g., Asgeirsson and Bjarnason Comp. Biochem. Physiol., Part B: Biochem. Mol. Biol. 99, 327 (1991); Simon et al. Biochem. Biophys. Res. Commun. 280, 1367 (2001); and Klibanov Nature 409, 241 (2001)). One of the most useful, and certainly the most therapeutically relevant application of chymotrypsin, is as an enzyme replacement therapy (see, e.g., Graham N. Engl. J. Med. 296, 1314 (1977)). In humans, chymotrypsin is secreted by the pancreas and is active in the small intestine, where it breaks down proteins. Chymotrypsin replacement therapy is used to treat diseases where low levels of the enzyme are a symptom (see, e.g., Geokas Clin. Geriatr. Med. 1, 177 (1985)). During this therapy, exogenous chymotrypsin is delivered orally to the gastrointestinal (GI) tract. Unfortunately for exogenous chymotrypsin delivery, the stomach uses acid and proteases to rapidly breakdown proteins (including chymotrypsin) in order to facilitate amino acid nutrient absorption into the bloodstream. Unmodified chymotrypsin has been investigated as an enzyme replacement therapy to treat autism, cystic fibrosis, and exocrine pancreatic insufficiency (Webb Nat. Biotechnol. 28, 772 (2010); Trapnell et al. Pediatr. Pulm. 49, 406 (2014); Somaraju and Solis-Moya Cochrane Db. Syst. Rev. (2014); Lisowska et al. J. Cystic Fibrosis 5, 253 (2006); and Fieker Clin. Exp. Gastroenterol. 4, 55 (2011)).

The term “complete denaturation” refers to an irreversible change in the structure of a protein (e.g., the secondary, tertiary and/or quaternary structure of a protein). For example, exposure of a protein to acidic conditions below pH 3.0 may induce a change in the fold of a protein such that the protein is not capable of refolding upon subsequent exposure to an environment having a higher pH (e.g., a neutral pH).

As used herein, “native state” refers to the structure of a protein (e.g., the secondary, tertiary and/or quaternary structure of a protein) prior to partial or complete denaturation (e.g., induced by exposure to acidic conditions (e.g., a pH below 3.0)). Native state, as used in reference to an enzyme, refers to a catalytically-active conformation of the enzyme.

As used herein, “partially unfolded state” refers to a structural conformation of a protein wherein the protein fold is partially disrupted as compared to a protein in its native state, and the protein is not completely denatured. In some embodiments, a protein in a partially unfolded state has diminished or no activity (e.g., enzymatic activity) as compared to the protein in its native state.

As used herein, “half-life” refers to the time required for a measured parameter, such as the potency, activity and effective concentration of a protein (e.g., a therapeutic protein) to decrease by half of its original level. Thus, the parameter, such as potency, activity, or effective concentration of a polypeptide molecule is generally measured over time. A half-life can be measured in vitro or in vivo. For example, the half-life of a therapeutic protein can be measured in vitro by assessing its activity following incubation over increasing time under certain conditions. In another example, the half-life of a therapeutic protein can be measured in vivo following administration (e.g., oral administration) of the protein to a subject, followed by obtaining a sample from the subject to determine the concentration and/or activity of the protein in the subject.

As used herein, “enzymatic activity” refers to the activity of an enzyme of catalyzing a chemical reaction, and may be expressed quantitatively (e.g., as the number of moles of substrate converted per unit time).

As used herein, the term “specific activity” refers to a measure of the activity of an enzyme per milligram of total protein. Specific activity is also a measure of enzyme processivity, at a specific substrate concentration.

As used herein the term “antibody” refers to any immunoglobulin (Ig) molecule comprised of four polypeptide chains, two heavy (H) chains and two light (L) chains, or any functional fragment, mutant, variant, or derivation thereof. Such mutant, variant, or derivative antibody formats are known in the art. In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG1, IgG2, IgG3, IgG4, IgA1 and IgA2) or subclass. In some embodiments, the antibody is a full-length antibody. In some embodiments, the antibody is a murine antibody, a human antibody, a humanized antibody, or a chimeric antibody. Exemplary functional fragments of an antibody include (i) a Fab fragment, a monovalent fragment consisting of the VL, VH, CL and CHI domains; (ii) a F(ab′)2 fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb fragment, which comprises a single variable domain; and (vi) an isolated complementarity determining region (CDR).

As used herein, “mucoadhesive” refers to the ability of a biomolecule (e.g., a protein, polymer, protein-polymer conjugate) to adhere to mucosa. In some embodiments, the mucoadhesion is mediated by the interaction of a biomolecule with a mucin protein. In vitro and in vivo methods of measuring the mucoadhesiveness are known in the art, and include, but are not limited to, the Wilhelmy plate method, the peel test, BIACORE assays, immunofluorescence labeling using protein-specific antibodies; staining of polymers using dyes; protein-labeling and detection in vivo, (see, e.g., Takeuchi et al. (2005) Adv. Drug. Deliv. Rev. 57: 1583-94; Kremser et al. (2008) Magnetic Resonance Imaging 26: 638-43; Kockisch et al. (2001) J. Control. Release 77: 1-6; Yu et al. (2014). Mucoadhesion and characterization of mucoadhesive properties, in Mucosal Delivery of Biopharmaceuticals, eds. das Neves J., Sarmento B., editors. (Boston, Mass.: Springer US), pp. 35-58; each of which are incorporated herein by reference)

As used herein, the term “gastrointestinal tract” refers to all portions of an organ system responsible for the consumption and digestion of foodstuffs, including the absorption of nutrients, and the expulsion of waste. The gastrointestinal tract includes orifices and organs such as the mouth, throat, esophagus, stomach, small intestine, large intestine, rectum, anus, sphincter, duodenum, jejunum, ileum, ascending colon, transverse colon, and descending colon, as well as the various passageways connecting the aforementioned portions.

As used herein, “specifically binds” or “specific binding” means that one biomolecule, such as a protein-polymer conjugate, binds preferentially a target (e.g., another biomolecule, such as a mucin), in the presence of other molecules. Specific binding can be influenced by, for example, the affinity and avidity of the biomolecule and the concentration of the biomolecule. In some embodiments, the biomolecule binds its target or specific binding partner with at least 2-fold greater affinity, and preferably at least 10-fold, 20-fold, 50-fold, 100-fold or higher affinity than it binds a non-specific molecule.

As used herein, the term “polymer length” refers to the length of the polymer as a result of the average number of monomer residues incorporated in a polymer chain. A “monomer” is a molecule that may bind chemically and covalently to other molecules to form a polymer.

As used herein, the term “protein” refers to a polypeptide (i.e., a string of at least two amino acids linked to one another by peptide bonds). A protein may include moieties other than amino acids, such as post-translational modifications and/or may be otherwise processed or modified. A protein can be a complete polypeptide chain as produced by a cell (with or without a signal sequence), or can be a functional portion thereof. In some embodiments, a protein can sometimes include more than one polypeptide chain, for example linked by one or more disulfide bonds or associated by other means.

As used herein, a “protein-polymer conjugate” refers to a protein that has been covalently modified to graft a polymer from functional groups present on the surface of the protein. In some embodiments, the protein-polymer conjugate comprises a protein that is partially unfolded. In some embodiments, the protein-polymer conjugate comprises a protein having a catalytically-active conformation.

As used herein the term “enzyme” refers to any of a group of catalytic proteins that are produced by native or transgenic living cells or protein engineering, and that mediate and/or promote a chemical processes or reaction. Enzymes show considerable selectivity for the molecules upon which they act (i.e., substrates). As used herein, the terms “active site” and “enzyme active site” refers to a specific region of an enzyme where a substrate binds and catalysis takes place (also referred to as “binding site”).

Protein-Polymer Conjugates

The protein-polymer conjugates described herein can be generated using polymerization processes that comprise polymerizing monomers under controlled polymerization processes in the presence of a complex comprising a monomer. In general two methods can be utilized to form polymeric chains extending from a protein: a “grafting-from” approach of a “grafting-to” approach.

In some embodiments, a protein-polymer conjugate can be formed using a “grafting-from” approach to polymerize a first plurality of first monomers on a polymerization initiator, resulting in a first polymeric chain being covalently bonded to the substrate. The “grafting-from” approach involves formation of the polymeric chain onto the protein surface. In this method, the polymerization of the polymeric chain can be conducted through any suitable type of free radical polymerization, such as reversible addition-fragmentation chain transfer (RAFT) polymerization, atom transfer radical polymerization (ATRP), etc.

In some embodiments, the polymer in the protein-polymer conjugate can be formed using a “grafting-to” approach whereby the polymeric chain is first polymerized and subsequently covalently bonded to the surface of the protein via a polymerization initiator.

In some embodiments, upon attachment, the polymeric chain can be deactivated to prevent further polymerization thereon. For example, if a “grafting-from” method is utilized to generate the protein-polymer conjugate (e.g., via ATRP), a deactivation agent can be utilized, e.g., attached to the end of each polymeric chain, to inhibit further polymerization thereon. Suitable deactivation agents can be selected based upon the type of polymerization and/or the type(s) of monomers utilized and include, but are not limited to, amines, peroxides, or mixtures thereof. If a “grafting-to” approach is used, the polymeric chain can be deactivated either prior to or after covalently bonding the polymeric chain to a polymerization initiator.

In some embodiments, the polymerization is performed under controlled radical polymerization conditions. A controlled radical polymerization (“CRP”) process is a process performed under controlled polymerization conditions with a chain growth process by a radical mechanism, such as, but not limited to, atom transfer radical polymerization, stable free radical polymerization, such as, nitroxide mediated polymerization, reversible addition-fragmentation transfer/degenerative transfer/catalytic chain transfer radical systems. In some embodiments, the polymerization process is performed by polymerizing monomers in the presence of at least one monomer and a transition metal. CRP processes are generally known to those skilled in the art and include atom transfer radical polymerization (“ATRP”), stable free radical polymerization (“SFRP”) including nitroxide mediated polymerization (NMP), and reversible addition-fragmentation chain transfer (“RAFT”). All three CRP processes are performed under conditions that maintain an equilibrium between a dormant species and an active species. The dormant species is activated with the rate constant of activation and form active propagating radicals. Monomer may react with the initiator or polymer chain as the active propagating radical. The propagating radicals are deactivated with the rate constant of deactivation (or the rate constant of combination) or may terminate with other growing radicals with the rate constant of termination. This equilibrium controls the overall polymerization rate.

In some embodiments, the protein-polymer conjugate is generated using ATRP. In ATRP, polymerization control is achieved through an activation-deactivation process, in which most of the reaction species are in dormant format, thus significantly reducing chain termination reaction. The four major components of ATRP include the monomer, initiator, ligand, and catalyst. The catalyst can determine the equilibrium constant between the active and dormant species during polymerization, leading to control of the polymerization rate and the equilibrium constant. The deactivation of radicals in ATRP includes reversible atom or group transfer that can be catalyzed by transition-metal complexes (e.g., transition metal complexes of Cu, Fe, Ru, Ni, Os, etc.). An initiator (e.g., alkyl halide, such as an alkyl bromide) can be activated by a transition metal complex to generate a radical species. Monomers can then be reacted with the radical species to attach monomer to the species (e.g., a protein of interest). The attached monomer can then be activated to form another radical and the process repeated with additional monomers, resulting in the generation of polymerized species. ATRP methods and improvements thereto are known in the art (see, e.g., U.S. Pat. Nos. 5,763,546; 5,807,937; 5,789,487; 5,945,491; 6,111,022; 6,121,371; 6,124,411; 6,162,882; 6,624,262; 6,407,187; 6,512,060; 6,538,091; 6,541,580; 6,624,262; 6,627,314; 6,759,491; 6,790,919; 6,887,962; 7,019,082; 7,049,373; 7,064,166; 7,125,938; 7,157,530; 7,332,550; 7,407,995; 7,572,874; 7,678,869; 7,795,355; 7,825,199; 7,893,173; 7,893,174; 8,252,880; 8,273,823; 8,349,410; 8,367,051; 8,404,788; 8,445,610; 8,865,797; 8,445,610; 8,871,831; 8,962,764; 9,664,042; U.S. Publication Nos. 2012/0213986; 2013/0131278; 2016/0200840; and 2017/0113934; International Publication Nos. WO 2016/130677, and WO 2015/051326; Matyjaszewski et al. ACS Symp. Ser. 685, 258-83 (1998); ACS Symp. Ser. 713, 96-112 (1998); ACS Symp. Ser. 729, 270-283 (2000); ACS Symp. Ser. 765, 52-71 (2000); ACS Symp. Ser. 768, 2-26 (2000); ACS Symposium Series 854, 2-9 (2003); ACS Symp. Ser. 1023, 3-13 (2009); ACS Symp. Ser. 1100, 1 (2012); Chem. Rev. 101, 2921-2990 (2001); and Progress in Polymer Science 32(1): 93-146 (2007), the entire contents of each of which are incorporated herein by reference in their entirety.

In some embodiments, the protein-polymer conjugate is generated using RAFT. RAFT polymerization uses thiocarbonylthio compounds (e.g., dithioesters, dithiocarbamates, trithiocarbonates, and xanthates) to mediate the polymerization via a reversible chain-transfer process. RAFT polymerization systems typically include a monomer, an initiator, and a RAFT agent (also referred to as a chain transfer agent). The polymerization reaction is started by the radical initiator, which reacts with a monomer unit to create a radical species thereby starting an active polymerizing chain. Then, the active chain reacts with the thiocarbonylthio compound of the RAFT agent, which expels a homolytic leaving group. The leaving group radical then reacts with another monomer species, starting another active polymer chain. RAFT polymerization allows the synthesis of polymers with specific macromolecular architectures such as block, gradient, statistical, comb/brush, star, hyperbranched, and network copolymers. Common RAFT agents contain thiocarbonyl-thio groups, and include, for example, dithioesters, dithiocarbamates, trithiocarbonates and xanthenes. RAFT methods and improvements thereto are known in the art (see, e.g., U.S. Pat. Nos. 8,865,796 and 9,359,453; U.S. Patent Application Publication No. 2015/0266990; and Grover and Maynard (2010) Current Opinion in Chemical Biology, 14(6), 818-827; Pelegri-O'Day and Maynard (2016) Accounts of Chemical Research, 49(9), 1777-1785; and Moad et al., The Chemistry of Radical Polymerization, 2d Ed., pp. 508 to 539, Elsevier (2006), the entire contents of the foregoing are incorporated herein by reference.)

In some embodiments, the protein-polymer conjugate is generated using SFRP. SFRP, and in particular NMP, achieves control of polymerization with a dynamic equilibrium between dormant alkoxyamines and actively propagating radicals. The use of nitroxides to mediate (i.e., control) free radical polymerization has been well studied and many different types of nitroxides have been described. Examples of useful NMP agents include those described in “The Chemistry of Radical Polymerization”, Moad et al., The Chemistry of Radical Polymerization, 2d Ed., pp. 473 to 475, Elsevier (2006), which is incorporated by reference herein.

In some embodiments, the at least one polymer of the protein-polymer conjugate is a positively-charged polymer. In some embodiments, the positively charged polymer is a quaternary ammonium polymer of Formula (I), wherein R₁ is H or CH₃; R₂ is O or NH; n is 2, 3, 4, or 5; and R3 is an alkyl. In some embodiments, the positively-charged polymer is a poly(N-alkl vinylpyridine) of Formula II, wherein R is an alkyl. In some embodiments, the positively-charged polymer is poly(quaternary ammonium methacrylate)(pQA).

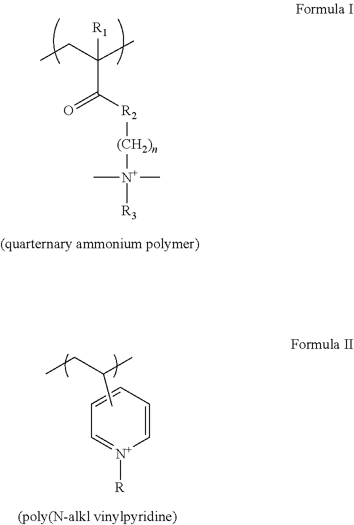

In some embodiments, the at least one polymer of the protein-polymer conjugate is a zwitterionic polymer. In some embodiments, the zwitterionic polymer is a carboxybetaine or a sulfobetaine polymer of Formula III, wherein R₁ is H or CH₃; R₂ is O or NH; n is 2, 3, 4, or 5; M is 2 or 3; and R₃ is COO⁻ or SO₃⁻. In some embodiments, the zwitterionic polymer is a phosphorylcholine polymer of Formula IV, wherein R₁ is H or CH₃; R₂ is O or NH; and n is 2, 3, 4, or 5. In some embodiments, the zwitterionic polymer is poly(carboxybetaine acrylamide) (pCBAm), poly(carboxybetaine methacrylate) (pCBMA), or poly(sulfobetaine methacrylamide) (pSBAm).

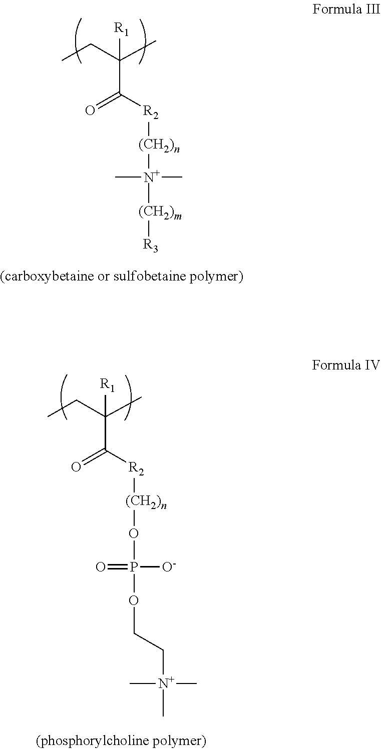

In some embodiments, the at least one polymer is mucoadhesive. In some embodiments, the at least one polymer specifically binds to mucin. The inclusion of a mucoadhesive polymer in a protein-polymer conjugate confers the conjugate the ability to bind to mucin. The ability of a protein-polymer conjugate to bind to mucin may be particularly advantageous in therapeutic applications in order to target the conjugate to a gastrointestinal tissue having mucin. In some embodiments, the conjugated protein of the protein-polymer conjugate does not bind to mucin in its native state (e.g., as an unconjugated protein).

In some embodiments, the mucoadhesive polymer is a positively-charged polymer. In some embodiments, the positively charged polymer is a quaternary ammonium polymer of Formula (I), wherein R1 is H or CH3; R2 is O or NH; n is 2, 3, 4, or 5; and R3 is an alkyl. In some embodiments, the positively-charged polymer is a poly(N-alkyl vinylpyridine) of Formula II, wherein R is an alkyl. In some embodiments, the positively-charged polymer is poly(quaternary ammonium methacrylate)(pQA). In some embodiments, the mucoadhesive polymer is a zwitterionic polymer. In some embodiments, the zwitterionic polymer is a carboxybetaine or a sulfobetaine polymer of Formula III, wherein R1 is H or CH3; R2 is O or NH; n is 2, 3, 4, or 5; M is 2 or 3; and R3 is COO— or SO3-. In some embodiments, the zwitterionic polymer is a phosphorylcholine polymer of Formula IV, wherein R1 is H or CH3; R2 is O or NH; and n is 2, 3, 4, or 5. In some embodiments, the zwitterionic polymer is poly(carboxybetaine acrylamide) (pCBAm), poly(carboxybetaine methacrylate) (pCBMA), or poly(sulfobetaine methacrylamide) (pSBAm). In some embodiments, the polymer is not poly(sulfobetaine methacrylamide)-block-poly(N-isopropylacrylamide).

In some embodiments, the protein-polymer conjugate may comprise at least one polymer exhibiting a polymer length ranging from a minimum of at least 2 monomer repeats to about 1000 monomer repeats. For example, the polymer length may range from at least about 5 monomer repeats to about 750 monomer repeats, from at least about 10 monomer repeats to about 200 monomer repeats, from at least about 10 monomer repeats to about 600 monomer repeats, from at least about 25 monomer repeats to about 500 monomer repeats, from at least about 50 monomer repeats to about 400 monomer repeats, from at least about 100 monomer repeats to about 250 monomer repeats, or any range subsumed therein. In some embodiments, the polymer length is from at least about 5 monomer repeats to about 150 monomer repeats. In some embodiments, the polymer length is from at least about 5 monomer repeats to about 200 monomer repeats.

In some embodiments, the protein-polymer conjugate composition may comprise a co-polymer comprising more than one monomeric repeating unit. In various aspects, the enzyme-polymer conjugate may comprise at least one polymer that is a co-polymer comprising at least two different monomers. In some embodiments, the co-polymer of the protein-polymer conjugate may comprise at least two different monomers, wherein at least one monomer may comprise a varied topology from at least one different monomer of the co-polymer. More specifically, the varied topology of the at least one monomer may include block, random, star, end-functional, or in-chain functional co-polymer topology. For example, at least one monomer of the co-polymer may include at least one monomer of a di-block topology. The co-polymers, monomers for di-block formation, monomers including an end functional group, or in-chain functional copolymers may be synthesized utilizing the materials and methods described in U.S. Pat. Nos. 5,789,487, and 6,624,263, U.S. Publication No. 2009/0171024, and Matyjaszewski and Davis, ed., Handbook of Radical Polymerization, John Wiley and Sons, Inc., Hoboken, N.J. (2002), the entire contents of the foregoing is incorporated herein by reference.

In some embodiments, the protein-polymer conjugate comprises a plurality of polymers. Generally, when ATRP is used to generate a protein-polymer conjugate described herein, any accessible amino group on the unconjugated protein surface can be modified to grow a polymer. In some embodiments, the protein-polymer conjugate comprises at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 125, at least 150, at least 175, at least 200, or more polymers.

In some embodiments, each polymer of the plurality of polymers comprises monomeric units of the same type. In some embodiments, the plurality of polymers comprises a first polymer and a second polymer, wherein the first polymer and the second polymer are each made of monomeric units of a different type. In some embodiments, the plurality of polymers comprises at least two polymers (e.g., 2, 3, 4, 5, 6, 7, 8, 9, 10, 15, 20, or more) each made of monomeric units of a different type. In some embodiments, the plurality of polymers comprises a first type of polymer and a second type of polymer, wherein the first type of polymer and the second type of polymer are each made of monomeric units of a different type. In some embodiments, the plurality of polymers comprises at least two types of polymers (e.g., 2, 3, 4, 5, 6, 7, 8, 9, 10, 15, 20, or more) each made of monomeric units of a different type. In some embodiments, the plurality of polymers comprises at least two types of polymers, each made of a different combination of monomeric units. In some embodiments, the plurality of polymers comprises a combination of at least one positively-charged polymer and at least one zwitterionic polymer. In some embodiments, the plurality of polymers comprises at least two positively-charged polymers. In some embodiments, the plurality of polymers comprises at least two zwitterionic polymers.

In some embodiments, the protein-polymer conjugates described herein are advantageously resistant to environments that are acidic (e.g., acidic solutions or gastric juice). The pH stabilization effect provided to a protein present in a protein-polymer conjugate described herein is particularly advantageous for therapeutic applications wherein a protein is administered orally to a subject. For instance, the oral delivery of therapeutically active proteins and peptides remains a challenge due, at least in part, to the strongly acidic environment of the stomach that denatures many therapeutic proteins. In healthy human subjects, the pH varies across segments of the gastrointestinal tract. While the pH of the gastric juices in the human stomach is very acidic (pH 1.5-3.5), the pH in the GI tract rapidly increases to about pH 6 in the duodenum. The pH then increases to about 7.4 in the terminal ileum, drops to about 5.7 in the caecum, and then gradually increases to about 6.7 in the rectum (see, e.g., Fallingborg Dan. Med. Bull. 46(3): 183-96). By stabilizing the protein against the denaturing effects of the highly acidic environment of the stomach, the protein-polymer conjugates described herein can be used to improve the delivery and absorption of therapeutic proteins to the lower gastrointestinal tract. In some embodiments, the conjugate of the protein-polymer conjugate stabilizes a partially unfolded state of the conjugated protein.

In some embodiments, the protein-polymer conjugate described herein is resistant to complete denaturation in an environment having a pH of about 3.0 or less (e.g., a pH of 2.9, 2.75, 2.5, 2.25, 2.0, 1.75, 1.5, 1.25, 1.0, 0.75, 0.5, or less). In some embodiments, the protein-polymer conjugate is resistant to complete denaturation in an environment having a pH of about 2.5 or less. In some embodiments, the protein-polymer conjugate is resistant to complete denaturation in an environment having a pH of about 2.0 or less. In some embodiments, the protein-polymer conjugates is resistant to complete denaturation in an environment having a pH of about 1.5 or less. In some embodiments, the protein-polymer conjugate is resistant to complete denaturation in an environment having a pH of about 1.0 or less.

In some embodiments, the conjugated protein undergoes conformational changes that do not alter the activity (e.g., enzymatic activity) of the protein when the conjugate is exposed to an environment having a pH of about 3.0 or less. In some embodiments, the conjugated protein undergoes a reversible conformational change that alter the activity (e.g., enzymatic activity) of the protein when the conjugate is exposed to an environment having a pH of about 3.0 or less, whereby upon subsequent exposure to a non-acidic environment (e.g., a pH of about 6.5 or more), the conjugated protein is capable of reverting to its native conformation thereby restoring its activity.

In some embodiments, the conjugated protein retains at least about 50% of its activity (e.g., enzymatic activity) when the conjugate is in an environment having a pH of about 3.0 or less (e.g., a pH of 2.9, 2.75, 2.5, 2.25, 2.0, 1.75, 1.5, 1.25, 1.0, 0.75, 0.5, or less. In some embodiments, the conjugated protein retains at least about 55%, 60%, 65%, 70%, 75%, 80%, 85%. 90%, 95%, 96%, 98%, or 99% of its activity (e.g., enzymatic activity) when the conjugate is exposed to a pH of about 3.0 or less. In some embodiments, the conjugated protein retains at least about 55%, 60%, 65%, 70%, 75%, 80%, 85%. 90%, 95%, 96%, 98%, or 99% of its activity (e.g., enzymatic activity) when the conjugate is exposed to a pH of about 2.5 or less. In some embodiments, the conjugated protein retains at least about 55%, 60%, 65%, 70%, 75%, 80%, 85%. 90%, 95%, 96%, 98%, or 99% of its activity (e.g., enzymatic activity) when the conjugate is exposed to a pH of about 2.0 or less. In some embodiments, the conjugated protein retains at least about 55%, 60%, 65%, 70%, 75%, 80%, 85%. 90%, 95%, 96%, 98%, or 99% of its activity (e.g., enzymatic activity) when the conjugate is exposed to a pH of about 1.5 or less. In some embodiments, the conjugated protein retains at least about 55%, 60%, 65%, 70%, 75%, 80%, 85%. 90%, 95%, 96%, 98%, or 99% of its activity (e.g., enzymatic activity) when the conjugate is exposed to a pH of about 1.0 or less.

In some embodiments, the conjugated protein has improved stability, activity and/or bioavailability as compared to the unconjugated protein from which the conjugate was derived. In some embodiments, the conjugated protein has a half-life of at least 125% of the half-life of the unconjugated protein in its native state when the conjugate exposed to an environment having a pH of about 3.0 or less. In some embodiments, the conjugated protein has a half-life of at least 150% of the half-life of the unconjugated protein in its native state when the conjugate is exposed to an environment having a pH of about 3.0 or less (e.g., a pH of 2.9, 2.75, 2.5, 2.25, 2.0, 1.75, 1.5, 1.25, 1.0, 0.75, 0.5, or less). In some embodiments, the conjugated protein has a half-life of at least 175% of the half-life of the unconjugated protein in its native state when the conjugate is exposed to an environment having a pH of about 3.0 or less (e.g., a pH of 2.9, 2.75, 2.5, 2.25, 2.0, 1.75, 1.5, 1.25, 1.0, 0.75, 0.5, or less). In some embodiments, the conjugated protein has a half-life of at least 200% of the half-life of the unconjugated protein in its native state when the conjugate is exposed to an environment having a pH of about 3.0 or less (e.g., a pH of 2.9, 2.75, 2.5, 2.25, 2.0, 1.75, 1.5, 1.25, 1.0, 0.75, 0.5, or less). In some embodiments, the conjugated protein has a half-life of at least 250% of the half-life of the unconjugated protein in its native state when the conjugate is exposed to an environment having a pH of about 3.0 or less (e.g., a pH of 2.9, 2.75, 2.5, 2.25, 2.0, 1.75, 1.5, 1.25, 1.0, 0.75, 0.5, or less).

In some embodiments, one or more polymers of the protein-polymer conjugate stabilize a partially unfolded state of the protein that is triggered by exposure of the conjugate to an acidic environment. This stabilization effect allows the conjugated protein to refold into a conformation (e.g., a native state of the unconjugated protein) that restores the protein's activity (e.g., enzymatic activity) upon exposure of the conjugate to a less acidic conditions (e.g., a pH greater than 3.0). In some embodiments, the conjugated protein is capable of refolding to a native state when the conjugate is in an environment having a pH above about 3.0. In some embodiments, the conjugated protein is capable of refolding to a native state when the conjugate is in an environment having a pH above about 3.5. In some embodiments, the conjugated protein is capable of refolding to a native state when the conjugate is in an environment having a pH above about 4.0 (e.g., a pH of about 4.5, about 5.0, about 5.5, about 6.0, about 6.5, about 7.0, about 7.5, about 8.0, about 8.5, about 9.0). In some embodiments, the conjugated protein is capable of refolding to a native state when the conjugate is in an environment having a pH of from about 5.5 to about 8.5 (e.g., a pH from about 6.0 to about 8.5, from about 6.5 to about 8.5, from about 5.5 to about 8.0, from about 6.0 to about 8.0, from about 6.5 to about 8.0, from about 7.0 to about 8.0, from about 6.0 to about 7.5, from about 6.5 to about 7.0).

In some embodiments, the conjugated protein is resistant to complete denaturation in the stomach of a human subject. In some embodiments, the conjugated protein is capable of refolding to a native state after the conjugate traverses the stomach and reaches the lower gastrointestinal tract (e.g., after the conjugate reaches the small intestine, the large intestine, the rectum, the anus, the sphincter, the duodenum, the jejunum, the ileum, the ascending colon, the transverse colon, and/or the descending colon) of a subject. In some embodiments, the conjugated protein is resistant to protease degradation. In some embodiments, the conjugated protein is resistant to protease degradation in the GI tract (e.g., in the small intestine) of a subject.

The protein-polymer conjugate described herein may be generated using any protein, including, but not limited to therapeutic proteins and proteins used in industrial applications (e.g., xylanase in paper preparation). In some embodiments, the protein is a recombinant protein. In some embodiments, the protein is a therapeutic protein. Multiple therapeutic proteins for the treatment of a variety of diseases are known in the art and can be conjugated to form a protein-polymer conjugate as described herein (see, e.g., Dimitrov Methods Mol Biol. 2012; 899: 1-26, incorporated herein by reference). In some embodiments, the protein is an antibody (e.g., a monoclonal antibody or a fragment thereof), a Fc fusion protein, an enzyme, an anti-coagulation protein, a blood factor, a bone morphogenetic protein, a growth factor, an interferon, an interleukin, a thrombolytic agent, a protein or peptide antigen, and a hormone.

In some embodiments, the protein is an enzyme. In some embodiments, the enzyme is selected from the group consisting of lactase, xylanase, chymotrypsin, trypsin, and a gluten-degrading enzyme (e.g., Aspergillus niger prolyl endoprotease, Dipeptidyl peptidase-IV, and a Rothia mucilaginosa subtisilin such as ROTMU0001_0241 (C6R5V9_9MICC), ROTMU0001_0243 (C6R5W1_9MICC), and ROTMU0001_240 (C6R5V8_9MICC) (see, e.g., Wei et al. Am J Physiol Gastrointest Liver Physiol. 2016, 311(3): G571-G580)). In some embodiments, the enzyme is chymotrypsin.

In some embodiments, the protein is an antibody selected from the group consisting of muromonab-CD3 (anti-CD3 receptor antibody), abciximab (anti-CD41 7E3 antibody), rituximab (anti-CD20 antibody), daclizumab (anti-CD25 antibody), basiliximab (anti-CD25 antibody), palivizumab (anti-RSV (respiratory syncytial virus) antibody), infliximab (anti-TNFα antibody), trastuzumab (anti-Her2 antibody), gemtuzumab ozogamicin (anti-CD33 antibody), alemtuzumab (anti-CD52 antibody), ibritumomab tiuxeten (anti-CD20 antibody), adalimumab (anti-TNFα antibody), omalizumab (anti-IgE antibody), tositumomab-131I (iodinated derivative of an anti-CD20 antibody), efalizumab (anti-CD11a antibody), cetuximab (anti-EGF receptor antibody), golimumab (anti-TNFα antibody), bevacizumab (anti VEGF-A antibody), natalizumab (anti α4 integrin), efalizumab (anti-CD11a), cetolizumab (anti-TNFα antibody), tocilizumab (anti-IL-6R), ustenkinumab (anti IL-12/23), alemtuzumab (anti CD52), natalizumab (anti α4 integrin), and variants thereof.

In some embodiments, the protein is a Fc fusion protein selected from the group consisting of Arcalyst/rilonacept (IL1R-Fc fusion), Orencia/abatacept (CTLA-4-Fc fusion), Amevive/alefacept (LFA-3-Fc fusion), Anakinra-Fc fusion (IL-1Ra-Fc fusion protein), etanercept (TNFR-Fc fusion protein), FGF-21-Fc fusion protein, GLP-1-Fc fusion protein, RAGE-Fc fusion protein, ActRIIA-Fc fusion protein, ActRIIB-Fc fusion protein, glucagon-Fc fusion protein, oxyntomodulin-Fc-fusion protein, GM-CSF-Fc fusion protein, EPO-Fc fusion protein, insulin-Fc fusion protein, proinsulin-Fc fusion protein and insulin precursor-Fc fusion protein, and analogs and variants thereof.

In some embodiments, the protein is an anti-coagulation protein selected from the group consisting of tissue plasminogen activator, heparin and hirudin.

In some embodiments, the protein is a blood factor selected from the group consisting of Factor II, Factor V, Factor VII, Factor VIII, Factor IX, Factor X, Factor XI, Factor XIII, protein C, protein S, von Willebrand Factor and antithrombin III.

In some embodiments, the protein is a bone morphogenetic protein (BMP) selected from the group consisting of BMP-2, BMP-4, BMP-6, BP-7, and BMP-2/7,

In some embodiments, the protein is a growth factor selected from the group consisting of platelet derived growth factor (PDGF), epidermal growth factor (EGF), transforming growth factor-α (TGF-α), transforming growth factor-β (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor-α (TNA-α), and placental growth factor (PLGF).

In some embodiments, the protein is an interferon selected from the group consisting of interferon-α, interferon-β, interferon-λ1, interferon-λ2 and interferon-λ3.

In some embodiments, the protein is a thrombolytic agent selected from the group consisting of tissue plasminogen activators, antistreptase, streptokinase, and urokinase.

In some embodiments, the protein is selected from the group consisting of insulin, oxytocin, vasopressin, adrenocorticotrophic hormone, prolactin, luliberin, growth hormone, growth hormone releasing factor, parathyroid hormone, somatostatin, glucagon, interferon, gastrin, tetragastrin, pentagastrin, urogastroine, secretin, prosecretin, calcitonin, angiotensin, renin, glucagon-like peptide-1, and human granulocyte colony stimulating factor (GM-CSF).

In some embodiments, the protein is a viral antigen, a parasite antigen, or a bacterial antigen. In some embodiments, the bacterial antigen is derived from a bacterium selected from the group consisting of Bacillus anthraces, Bordetella pertussis, Campylobacter jejuni, Chlamydia pneumoniae, Clostridium botulinum, Clostridium difficile, Clostridium perfringens, Clostridium tetani, Corynebacterium diptheriae, Enterococcus faecalis, Enterococcus faecium, Escherichia coli, enterotoxigenic Escherichia coli, enteropathogenic Escherichia coli, Escherichia coli O157:H7, Francisella tularensis, Haemophilus influenza, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitides, Pseudomonas aeruginosa, Rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae, and Yersinia pestis. In some embodiments, the viral antigen is derived from a virus selected from the group consisting of adenovirus, arbovirus, astrovirus, coronavirus, Coxsackievirus, Crimean-Congo hemorrhagic fever virus, cytomegalovirus (“CMV”), dengue virus, ebola virus, Epstein-Barr virus (“EBV”), foot-and-mouth disease virus, Guanarito virus, Hendra virus, herpes simplex virus-type 1 (“HSV-1”), herpes simplex virus-type 2 (“HSV-2”), human herpesvirus-type 6 (“HHV-6”), human herpesvirus-type 8 (“HHV-8”), hepatitis A virus (“HAV”), hepatitis B virus (“HBV”), hepatitis C virus (“HCV”), hepatitis D virus (“HDV”), hepatitis E virus (“HEV”), human immunodeficiency virus (“HIV”), influenza virus, Japanese encephalitis virus, Junin virus, Lassa virus, Machupo virus, Marburg virus, Norovirus, Norwalk virus, human papillomavirus (“HPV”), parainfluenza virus, parvovirus, poliovirus, rabies virus, respiratory syncytial virus (“RSV”), rhinovirus, rotavirus, rubella virus, Sabia virus, severe acute respiratory syndrome virus (“SARS”), varicella zoster virus, variola virus, West Nile virus, and yellow fever virus. In some embodiments, the parasite antigen is derived from a parasite selected from the group consisting of Cryptosporidium spp., Cyclospora cayetanensis, Diphyllobothrium spp., Dracunculus medinensis, Entamoeba histolytica, Giardia duodenalis, Giardia intestinalis, Giardia lamblia, Leishmania sp., Plasmodium falciparum, Schistosoma mansoni, Schistosoma haematobium, Schistosoma japonicum, Taenia spp., Toxoplasma gondii, Trichinella spiralis, and Trypanosoma cruzi.

In some embodiments, the protein is a hormone selected from the group consisting of nerve growth factor (NGF), platelet derived growth factor (PDGF), fibroblast growth factor (FGF), calcitonin, cortistatin, endothelin, erythropoietin, gastrin, ghrelin, inhibin, osteocalcin, luteinizing hormone, oxytocin, prolactin, secretin, renin, somatostatin, thrombopoietin, and insulin.

In some embodiments, the protein is selected from the group consisting of amyloid β peptide (Aβ); α-synuclein, microtubule-associated protein tau (Tau protein), TDP-43, Fused in sarcoma (FUS) protein, superoxide dismutase, C9ORF72, ubiquilin-2 (UBQLN2), ABri, ADan, Cystatin C, Notch3, Glial fibrillary acidic protein (GFAP), Seipin, transthyretin, serpins, amyloid A protein, islet amyloid polypeptide (IAPP; amylin), medin (lactadherin), apolipoprotein AI, apolipoprotein AII, apolipoprotein AIV, Gelsolin, lysozyme, fibrinogen, beta-2 microglobulin, crystallin, rhodopsin, calcitonin, atrial natriuretic factor, prolactin, keratoepithelin, keratin, keratin intermediate filament protein, lactoferrin, surfactant protein C (SP-C), odontogenic ameloblast-associated protein, semenogelin I, apolipoprotein C2 (ApoC2), apolipoprotein C3 (ApoC3), leukocyte chemotactic factor-2 (Lect2), galectin-7 (Ga17), corneodesmosin, enfuvirtide, cystic fibrosis transmembrane conductance regulator (CFTR) protein, and hemoglobin.

Pharmaceutical Compositions

In another aspect, compositions comprising a protein-polymer conjugate described herein are also provided. In some embodiments, the composition is a foodstuff (e.g., a beverage or a solid foodstuff including a nutritional supplement). In some embodiments, the composition is a pharmaceutical composition.

Pharmaceutical compositions can be prepared according to any method known to the art for the manufacture of pharmaceuticals, and can include sweetening agents, flavoring agents, coloring agents and preserving agents. A pharmaceutical composition can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by a subject. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents (e.g., a protein-polymer conjugate) can be dissolved or suspended in suitable liquids, such as fatty oils, liquid paraffin, or liquid polyethylene glycol with or without stabilizers.

Aqueous suspensions can contain an active agent (e.g., a protein-polymer conjugate) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Method of Treatment

Methods of using the protein-polymer conjugate or pharmaceutical composition are also provided. In one aspect, provided herein are methods of enhancing the delivery of a protein (e.g., a therapeutic protein such an enzyme) to a subject by administering a protein-polymer conjugate or pharmaceutical composition comprising the protein-polymer conjugate. As described above, the protein-polymer conjugates described herein are particularly advantageous as they provide for the stability of the conjugated protein in environments having an acidic pH (e.g., a pH of about 3.0 or less). Upon administration to a subject (e.g., orally) the protein-polymer conjugate described herein allows for the conjugated protein to be protected against complete denaturation in the highly acidic environment of the stomach, e.g., by stabilizing a partially unfolded state of the protein. Without wishing to be bound by any particular theory, the resistance of the protein-polymer conjugates to the acidic environment of the stomach allows for a smaller concentration of conjugated protein (as compared to unconjugated protein) to be administered to a subject in order to achieve a desireable response (e.g., a therapeutic effect).

In some embodiments, mucoadhesive protein-polymer conjugates or a pharmaceutical composition comprising the mucoadhesive protein-polymer conjugates described herein can be used in methods to target the delivery of the conjugated protein to the gastrointestinal tract of the subject. Specifically, the mucoadhesive protein-polymer conjugates can be used to target a section of the gastrointestinal tract comprising mucin. In some embodiments, the mucoadhesive protein-polymer conjugates have a higher retention time at a particular tissue of the subject (e.g., the small intestine) allowing for the conjugated protein to be absorbed by the subject or to perform a particular function at the site of retention or mucoadhesion.

In some embodiments, a protein-polymer conjugate described herein exhibits reduced immunogenicity (e.g., a reduced humoral response or a reduced adaptive immune response) as compared to the unconjugated protein from which it was generated.

In some embodiments, provided herein are methods of treating a disease or disorder in a subject in need thereof, comprising administering a therapeutically-effective amount of the protein-polymer conjugate or pharmaceutical composition comprising the protein-polymer conjugate to the subject. One of ordinary skill will appreciate that the compositions described herein may be adapted for use with any protein (e.g., a therapeutic protein) in order to treat any disease or disorder, including, but not limited to, a proliferative disease or disorder (e.g., cancer), an infectious disease, an autoimmune disease, an inflammatory disease (e.g., Crohn's disease or rheumatoid arthritis), an allergy, a genetic disease or disorder, or a proteopathy. In some embodiments, the protein-polymer conjugates are used in enzyme replacement therapy. For example, in some embodiments, a protein-polymer conjugate comprising chymotrypsin is used for the treatment of a disease or disorder selected from the group consisting of autism, cystic fibrosis, and exocrine pancreatic insufficiency.

Proteopathies that may be treated using the methods provided herein, as well as proteins that may be used in their treatment (in parenthesis) include Alzheimer's disease (Amyloid β peptide (Aβ); Tau protein); cerebral β-amyloid angiopathy (amyloid β peptide (Aβ)); retinal ganglion cell degeneration in glaucoma (amyloid β peptide (Aβ)); Parkinson's disease and other synucleinopathies (α-Synuclein); tauopathies (microtubule-associated protein tau (Tau protein)); frontotemporal lobar degeneration (FTLD) (TDP-43); FTLD-FUS (Fused in sarcoma (FUS) protein); amyotrophic lateral sclerosis (ALS) (superoxide dismutase, TDP-43, FUS, C9ORF72, ubiquilin-2 (UBQLN2)); Huntington's disease and other trinucleotide repeat disorders (proteins with tandem glutamine expansions); familial British dementia (ABri); familial Danish dementia (ADan); hereditary cerebral hemorrhage with amyloidosis (Icelandic) (HCHWA-I) (cystatin C); CADASIL (Notch3); Alexander disease (Glial fibrillary acidic protein (GFAP)); seipinopathies (seipin); familial amyloidotic neuropathy and senile systemic amyloidosis (transthyretin); serpinopathies (serpins); AL (light chain) amyloidosis (primary systemic amyloidosis) (monoclonal immunoglobulin light chains); AH (heavy chain) amyloidosis (immunoglobulin heavy chains); AA (secondary) amyloidosis (Amyloid A protein); type II diabetes (Islet amyloid polypeptide (IAPP; amylin)); aortic medial amyloidosis (lactadherin); ApoAI amyloidosis (Apolipoprotein AI); ApoAII amyloidosis (Apolipoprotein AII); ApoAIV amyloidosis (Apolipoprotein AIV); familial amyloidosis of the Finnish type (FAF) (gelsolin); Lysozyme amyloidosis (lysozyme); fibrinogen amyloidosis (fibrinogen); dialysis amyloidosis (beta-2 microglobulin); inclusion body myositis/myopathy (amyloid β peptide (ADA cataracts (crystallins); retinitis pigmentosa with rhodopsin mutations (rhodopsin); medullary thyroid carcinoma (calcitonin); cardiac atrial amyloidosis (atrial natriuretic factor); pituitary prolactinoma (prolactin); hereditary lattice corneal dystrophy (keratoepithelin); cutaneous lichen amyloidosis (keratins); mallory bodies (keratin intermediate filament proteins); corneal lactoferrin amyloidosis (lactoferrin); pulmonary alveolar proteinosis (surfactant protein C (SP-C)); odontogenic (Pindborg) tumor amyloid (odontogenic ameloblast-associated protein); seminal vesicle amyloid (semenogelin I); apolipoprotein C2 amyloidosis (apolipoprotein C2 (ApoC2)); apolipoprotein C3 amyloidosis (apolipoprotein C3 (ApoC3)); lect2 amyloidosis (leukocyte chemotactic factor-2 (Lect2)); insulin amyloidosis (insulin); galectin-7 amyloidosis (primary localized cutaneous amyloidosis) (galectin-7 (Ga17)); corneodesmosin amyloidosis (corneodesmosin); enfuvirtide amyloidosis (enfuvirtide); cystic fibrosis (cystic fibrosis transmembrane conductance regulator (CFTR) protein); and sickle cell disease (hemoglobin).

While the compositions described herein are particularly advantageous for oral administration, the compositions may be administered to a subject using any desireable route, including, intraocularlly, intranasally, parenterally, intravenously, intravaginally, intradermally, or rectally. The amount of composition administered to a subject should be adequate to accomplish therapeutically efficacy dose. The dosage schedule and amounts effective for this use, i.e., the dosing regimen, will depend upon a variety of factors, including the stage of the disease or condition, the severity of the disease or condition, the general state of the subject's health, the subject's physical status, age and the like. In calculating the dosage regimen for a subject, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; and Remington: The Science and Practice of Pharmacy, 21st ed., (2005). The state of the art allows the clinician to determine the dosage regimen for each individual subject, active agent and disease or condition treated. Guidelines provided for similar compositions used as pharmaceuticals can be used as guidance to determine the dosage regimen, i.e., dose schedule and dosage levels, administered practicing the methods of the invention are correct and appropriate.

### EXAMPLES

The invention is further described in the following examples, which do not limit the scope of the invention described in the claims.

### Materials and Methods for Example 1

All chemicals were purchased from Sigma-Aldrich (St. Louis, Mo.) and used as received unless otherwise indicated. Poly(ethylene glycol) methyl ether methacrylate (Mₙ=475) (OEGMA475) was filtered through basic alumina column to remove inhibitor prior to use. Me6TREN (Ciampolini and Nardi Inorg. Chem. 1966, 5, 41) carboxybetaine acrylamide (Kostina et al. Biomacromolecules 2012, 13, 4164), and quaternary ammonium methacrylate (Cummings et al. Biomacromolecules 2014, 15, 763) were synthesized as described previously. Dialysis tubing (molecular weight cut off, 15 kDa, Spectra/Por®, Spectrum Laboratories Inc., CA) for conjugate isolation were purchased from Thermo Fisher Scientific (Waltham, Mass.).

Initiator Immobilization onto Chymotrypsin Synthesis of the ATRP initiating molecules was carried out as described previously (see Murata et al. Biomacromolecules 2013, 14, 1919). Following synthesis, the initiator molecule (NHS-Br) (469 mg, 1.4 mmol) and CT (1.0 g, 0.04 mmol protein, 0.56 mmol —NH₂ group in lysine residues) were dissolved in sodium phosphate buffer (100 mL, 0.1 M NaPhos (pH 8)). The solution was stirred at 4° C. for 3 hours, and then dialyzed against deionized water, using dialysis tubing with a molecular weight cut off of 15 kDa, for 24 hours at 4° C. and then lyophilized. Initiator immobilization was quantified using matrix assisted laser desorption ionization-time of flight mass spectrometry (MALDI-TOF-MS) on a PerSeptive Voyager STR MS with nitrogen laser (337 nm) and 20 kV accelerating voltage located at the CMA, CMU, Pittsburgh, Pa. using sinapinic acid as the matrix and a gold sample plate. MALDI-TOF MS instrumentation was supported by NSF grant CHE-9808188.

Surface Initiated ATRP from CT-Br

Chymotrypsin-pOEGMA and chymotrypsin-pSMA were synthesized using CuCl/CuCl₂/bpy in deionized water (Averick et al. ACS Macro Lett. 2012, 1, 6). For CT-pOEGMA, 4.6 mL of a deoxygenated CuCl/CuCl₂/bpy stock solution (5 mM/45 mM/110 mM) in deionized water was added to 16.4 mL of a CT-Br (50 mg, 1.4 mM initiator) and OEGMA475 (2415 mg, 330 mM, targeted degree of polymerization 225) solution in deoxygenated deionized water and allowed to react at 4° C. for 80 minutes. CT-pSMA was synthesized by adding 4.6 mL of stock CuCl/CuCl₂/bpy (5 mM/45 mM/110 mM) in deoxygenated deionized water to 16.4 mL of CT-Br (50 mg, 1.4 mM initiator) and SMA (1190 mg, 285 mM, targeted degree of polymerization 227) in deoxygenated 100 mM NaPhos (pH 7) and allowed to react for 65 minutes at 4° C. CT-pQA was synthesized by adding 2 mL of CuBr (3.7 mg, 16 mM) and HMTETA (7.4 mg, 16 mM) in deoxygenated deionized water to 25 mL of CT-Br (50 mg, 1.4 mM initiator) and QA monomer (405 mg, 64 mM) in 64 mM deoxygenated NaSO₄ solution and allowed to react at 25° C. for 120 minutes (Murata et al. Biomacromolecules 2014, 15, 2817). Lastly, CT-pCBAm was synthesized by adding 5 mL of CT-Br (50 mg, 1.4 mM initiator) and CBAm (348 mg, 332 mM) in deoxygenated 100 mM NaPhos (pH 7) buffer to 2 mL of CuCl (2.5 mg, 12 mM) and Me6TREN (5.5 mg, 12 mM) in deoxygenated deionized water and allowed to react for 120 minutes at 4° C. (Millard et al. In Controlled/Living Radical Polymerization: Progress in ATRP; American Chemical Society: 2009; Vol. 1023, p 127). All conjugates were purified using dialysis tubing (MWCO 25 kDa) against deionized water for 48 hours at 4° C. Samples were lyophilized and chymotrypsin weight percent in each conjugate was determined using BCA assay.

Molecular Characterization of Conjugates and Polymers

All polymers were cleaved from the surface of CT-polymer conjugates using acid hydrolysis. CT conjugates (15 mg/mL) were incubated in 6N HCl at 110° C. under vacuum for 24 hours. Following incubation, cleaved polymers were isolated from CT using dialysis tubing (MwCO 1K Da) for 48 hours and then lyophilized. Number and weight average molecular weights (Mₙ and M_{w}) and the polydispersity index (M_{w}/Mₙ) were estimated by gel permeation chromatography (GPC) for polymers cleaved from CT. Analysis was conducted on a Waters 2695 Series with a data processor, using 0.1 M sodium phosphate buffer (pH 7.0) with 0.01 volume % NaN₃ (pOEGMA, pCBAm), 0.1 M sodium phosphate (pH 2.0) with 0.5% TFA (pQA), or 80% sodium phosphate (pH 9.0)/20% acetonitrile (pSMA) as eluent at a flow rate 1 mL/min, with detection by a refractive index (RI) detector, and PEG (pOEGMA, pCBAm, pQA) or polystyrene sulfonate (pSMA) narrow standards for calibration.

A Micromeritics (Norcross, Ga.) NanoPlus 3 dynamic light scattering (DLS) instrument was used to measure the intensity average hydrodynamic diameter (Dₕ) of each of the chymotrypsin conjugates at 2 mg/mL in 50 mM NaPhos (pH 7) buffer at 25° C. Histograms of results were plotted after 70 accumulation times, and average Dₕ values were calculated from these runs.

In Vitro Polymer Mucoadhesion

Mucoadhesion of free polymers was evaluated using mucin in different buffer systems. Free polymers were synthesized by the same protocol as for CT conjugates, but with a small molecule initiator instead of the chymotrypsin macroinitiator. Polymers were dissolved at 1 mg/mL in different buffers (167 mM HCl (pH 1), 50 mM ammonium acetate (pH 4.5), 50 mM NaPhos (pH 8)) and mixed with mucin protein (3 mg/mL in deionized water) at different weight ratios. After mixing, solutions were incubated for 30 minutes at 37° C. and absorbance at 400 nm (turbidity) was recorded. Turbidity measurements were plotted as relative ratios to the turbidity measurement at w/w ratio 0.0. For experiments with NaCl and ethanol, polymers were dissolved in buffer solutions with either 0.2 M NaCl, 0.5 M NaCl, or 10% v/v ethanol and then mixed with mucin.

Free polymer zeta potential 0 values were measured on a Micromeritics (NanoPlus 3) zetasizer instrument. Free polymers were dissolved at 2 mg/mL in specified buffer s3.6olution. Zeta potential values were averages of 4 repeat runs.

CT Conjugate Biocatalytic Activity

N-succinyl-L-Ala-L-Ala-L-Pro-L-Phe-p-nitroanilide was used as a substrate for enzyme bioactivity assays. In a cuvette, 0.1 M sodium phosphate buffer (930-990 μL, pH 6, 7, or 8), substrate (0-60 μL, 6 mg/mL in DMSO), and enzyme (10 μL, 0.1 mg enzyme/mL 0.1 M pH 8.0 sodium phosphate buffer (4 μM)) were mixed at 37° C. using a circulating water bath. The rate of the hydrolysis was determined by recording the increase in absorbance at 412 nm for the first 30 seconds after mixing. K_{M} and k_{cat} values were calculated using Graphpad software with Michaelis-Menten curve fit when plotting substrate concentration versus the initial rate for substrate hydrolysis.

In Vitro Gastric Acid Stability

Native CT and CT-conjugates were incubated at 4 μM in 167 mM HCl at 37° C. in 50 μL aliquots. Aliquots were removed at specified time points and residual activity was measured at 37° C. in 0.1 M sodium phosphate buffer (pH 8.0) with Suc-AAPF-pNA as substrate (288 μM). Each time point was measured in triplicate and residual activity was calculated as the ratio of activity remaining from time zero.

Intrinsic Tryptophan Fluorescence of CT Conjugates

CT conjugates were incubated at 37° C. in 167 mM HCl (pH 1) at 12 μM CT in 100 μL aliquots for each time point. At the specified time point, samples were diluted to 4 μM using 0.1 M NaPhos buffer (pH 8) and the intrinsic fluorescence was measured in triplicate at 37° C. Spectrum emission from 300-400 nm was measured for each sample after excitation at 270 nm. The wavelength values corresponding to the maximum emission intensity for each measurement were calculated and the average maximum wavelength (λₘₐₓ) was plotted for each sample.

Surface Charge Analysis of CT Initiator Complex

The initial structure of CT-Initiator complex was built with Maestro built toolkit (Schrodinger) using the crystal structure of CT from the Protein Data Bank (PDB ID 1YPH) as the starting structure. To remove the bias and constraints of the starting point, the structure was subjected to a Simulated Annealing (SA) protocol using Desmond (Bowers et al. In SC 2006 Conference, Proceedings of the ACM/IEEE; IEEE: 2006, p 43). This annealing protocol consisted of three stages with 100, 300, and 600 ps durations and temperature intervals from 300-400 K, 450-300 K, and 300 K, respectively. The simulation system was prepared using Desmond's system builder with the OPLS-2005 force field and SPC was chosen as a solvent model. An orthorhombic shape was chosen for the simulation box and its volume minimized with Desmond tool with no ions added to neutralize the system. NVT ensemble and the Berendsen thermostat method were used for temperature coupling with a relaxation time of 1 ps. A cutoff of 9 Å for van der Waals interactions was applied, and the particle mesh Ewald algorithm was used for Coulomb interactions with a switching distance of 9 Å. The total simulation time was 1 ns with recording interval energy 1.2 ps and recording trajectory of 5 ps. The final structure obtained after SA was then subjected molecular dynamics simulation (MD). Finally, a 10 ns MD simulation was performed using Desmond at 300 K with a time-step bonded of 2 fs. Trajectory energy values were recorded every 1.2 ps and structure energy was recorded every 4.8 ps. NPT ensemble, the ‘Nose-Hoover chain’ thermostat, and ‘Martyna-Tobia-Klein’ Barostat methods were used with 2 ps relaxation time and isotropic coupling. The default relaxation model, a cutoff of 9 Å for van der Waals interactions, and 200 force constant restrain one atom from the backbone were applied. The particle mesh Ewald algorithm was used for Coulomb interactions with a switching distance of 9 Å and no ions were added to the solution.

Predicted ionization states of chymotrypsin-initiator complex at neutral pH and pH 1 were determined using PROPKA 2.0 (Bas et al. Proteins: Struct., Funct., Bioinf. 2008, 73, 765). Surface charge analysis and molecular graphics for CT-Br were obtained using electrostatic potential coulombic surface coloring in UCSF Chimera package (Pettersen et al. J. Comput. Chem. 2004, 25, 1605).

### Materials and Methods for Example 2

Materials

α-Chymotrypsin (CT) from bovine pancreas (type II) was purchased from Sigma Aldrich (St Louis, Mo.). Protein surface active ATRP initiator (NHS-Br initiator) was prepared as described previously (Murata Biomacromolecules 14, 1919-1926 (2013)). All materials were purchased from Sigma Aldrich (St Louis, Mo.) and used without further purification unless stated otherwise. Dialysis tubing (Spectra/Por, Spectrum Laboratories Inc., CA) was purchased from ThermoFisher (Waltham, Mass.).

Initiator modification and characterization Initiator modified CT (CTBr) was synthesized by reacting NHS-Br (469 mg, 1.4 mmol) and CT (1.0 g, 0.04 mmol protein, 0.56 mmol primary amines) in 100 mM sodium phosphate buffer (pH 8, 100 mL). The solution was stirred at 4° C. for 3 hours, then dialyzed against deionized water (MWCO 15 kDa) overnight, then lyophilized.

CTBr was characterized using MALDI-ToF MS. MALDI-ToF-MS measurements were recorded using a PerSeptive Voyager STR MS with nitrogen laser (337 nm) and 20 kV accelerating voltage with a grid voltage of 90%. 300 laser shots covering the spot were accumulated for each spectrum. The matrix was composed of sinapinic acid (20 mg/mL) in 50% acetonitrile with 0.4% trifluoroacetic acid. Protein solutions of native CT and CTBr (1.0 mg/mL) were mixed with an equal volume of matrix and 2 μL of the resulting mixture was spotted on a sterling silver target plate. Apomyoglobin, cytochrome C, and aldolase were used as calibration samples. Number of initiator modifications was determined by taking the difference in peak m/z between native CT and CTBr and dividing by the molecular weight of the initiator (220.9 Da).

ATRP from Initiator Modified Sites

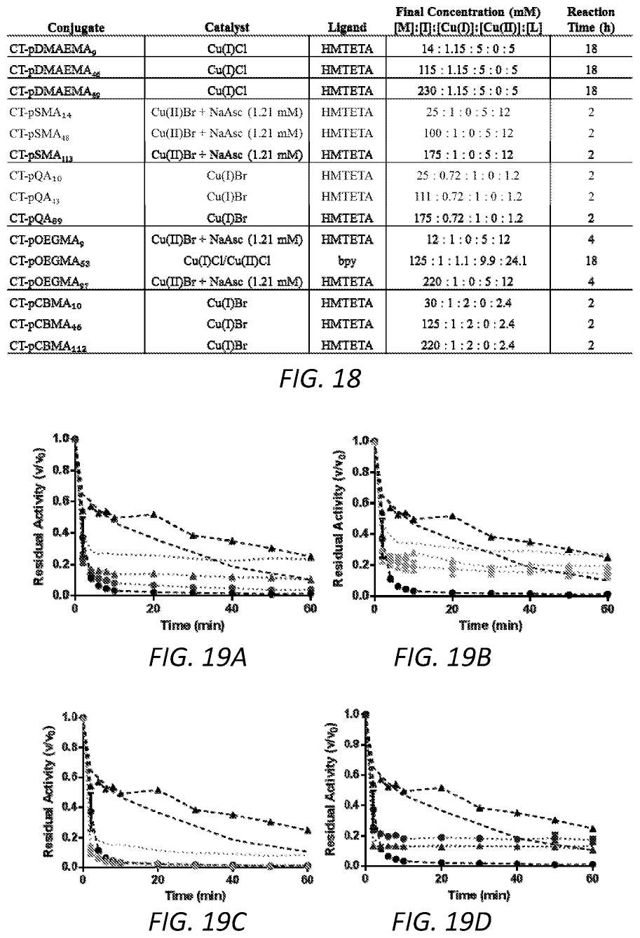

Copper (I) bromide (Cu(I)Br), copper (II) bromide (Cu(II)Br), copper (I) chloride (Cu(I)Cl), copper (II) chloride (Cu(II)Cl), sodium ascorbate (NaAsc) 1,1,4,7,10,10-Hexamethyltriethylenetetramine (HMTETA), and 2,2′-Bypyridyl(bpy) were purchased from Sigma Aldrich. HMTETA was purified prior to use using a basic alumina column. A summary of ATRP reaction conditions are provided in FIG. 18. After the reaction stopped via exposure to air, all conjugates were purified using dialysis (MWCO 25 kDa) against deionized water for 48 h at 4° C. followed by lyophilization. Lengths were varied by increasing the target degree of polymerization (DP) by increasing the monomer to initiator ratio.

Synthesis of CT-pCBMA

3-[[2-(Methacryloyloxy) ethyl] dimethylammonio] propionate (CBMA) was purchased from TCI America. CT-pCBMA was synthesized by adding CTBr (50 mg, 4.7 mg initiator) and CBMA (dependent on target DP) to 16.4 mL of 100 mM sodium phosphate buffer (pH 8). This solution was stirred on ice and bubbled under argon for 30 minutes to deoxygenate the system. In a separate flask, Cu(I)Br (6.02 mg) was added to 4.6 mL of deionized water and the solution bubbled under argon for 30 minutes with HMTETA (13.7 μL). The 4.6 mL of catalyst solution was added to the CBMA/CTBr solution. The reaction was stirred at 4° C. for 2 hours. Target DPs were 30, 125, and 220 for short, medium, and long length conjugates.

Synthesis of CT-pOEGMA

Poly(ethylene glycol) methyl ether methacrylate (Mₙ=500, OEGMA) was filtered through basic alumina column to remove inhibitor prior to use. CT-pOEGMA was synthesized by adding CTBr (50 mg, 4.7 mg initiator) and OEGMA (dependent on target DP) to 16.4 mL of deionized water. This solution was stirred on ice and bubbled under argon for 30 minutes to deoxygenate the system. In a separate flask, Cu(II)Br (23.45 mg) was added to 4.6 mL of deionized water and the solution bubbled under argon for 30 minutes with HMTETA (68.5 NaAsc (5 mg) was added to the catalyst solution, then the 4.6 mL of catalyst solution was added to the OEGMA/CTBr solution. The reaction was stirred at 4° C. for 4 hours. Target DPs were 12 and 220 for short and long length conjugates, respectively. The medium length conjugate was synthesized using a catalyst solution of Cu(I)Cl/Cu(II)Cl/bpy (5 mM/45 mM/110 mM) and was reacted for 18 hours (target DP=125). All other conditions were similar to the synthesis of short and long conjugates.

Synthesis of CT-pDMAEMA

(Dimethylamino) ethyl methacrylate (DMAEMA) was filtered through a basic alumina column prior to use. CT-pDMAEMA was synthesized by adding CTBr (50 mg, 4.7 mg initiator) and DMAEMA (dependent on target DP) to 15 mL of deionized water. This solution was stirred on ice and bubbled under argon for 30 minutes to deoxygenate the system. In a separate flask, Cu(I)Cl (10 mg) was added to 5 mL of deionized water and the solution bubbled under argon for 30 minutes with HMTETA (27.5 μL). The 5 mL of catalyst solution was added to the DMAEMA/CTBr solution. The reaction was stirred at 4° C. for 18 hours. Target DPs were 12, 100, and 200 for short, medium, and long length conjugates.

Synthesis of CT-pQA

Quaternary ammonium methacrylate (QA) was synthesized as previously described (Murata et al. Biomacromolecules 15, 2817-2823 (2014)). CT-pQA was synthesized by adding CTBr (50 mg, 4.7 mg initiator) and QA (dependent on target DP) to 25 mL of 64 mM sodium sulfate buffer (pH 8). This solution was stirred on ice and bubbled under argon for 30 minutes to deoxygenate the system. In a separate flask, Cu(I)Br (3.7 mg) was added to 2 mL of deionized water and the solution bubbled under argon for 30 minutes with HMTETA (8.74 μL). The 2 mL of catalyst solution was added to the QA/CTBr solution. The reaction was stirred at 4° C. for 2 hours. Target DPs were 35, 154, and 243 for short, medium, and long length conjugates.

Synthesis of CT-pSMA

3-sulfopropyl methacrylate potassium salt (SMA) was purchased through Sigma Aldrich. CT-pSMA was synthesized by adding CTBr (50 mg, 4.7 mg initiator) and SMA (dependent on target DP) to 16.4 mL of 100 mM sodium phosphate buffer (pH 8). This solution was stirred on ice and bubbled under argon for 30 minutes to deoxygenate the system. In a separate flask, Cu(II)Br (23.45 mg) was added to 4.6 mL of deionized water and the solution bubbled under argon for 30 minutes before NaAsc (5 mg) was added. After addition of HMTETA (68.5 μL), the 4.6 mL of catalyst solution was added to the SMA/CTBr solution. The reaction was stirred at 4° C. for 2 hours. Target DPs were 25, 100, and 175 for short, medium, and long length conjugates.

Prediction of log D and pKa

ChemAxon was used to calculate the hydrophobicity (log D) and pKₐ of the monomers. The pKₐ was estimated from the inflection point of the log D versus pH plot. This is the point at which the protonation state changes as evidenced by a sharp change in log D.

Conjugate Determination of Protein Content

Protein content of CT-conjugates was determined in triplicate using a bichinchoninic acid (BCA) assay according to Sigma Aldrich microplate protocol. Briefly, 0.5-1.0 mg/mL of CT-conjugates were prepared in deionized water along with native CT standards. To each well, 25 μL of sample was added to 200 μL of working solution (1:8 ratio). The plate was covered and incubated at 37° C. for 30 minutes followed by measuring the absorbance at 562 nm using a BioTek Synergy H1 Plate Reader. The degree of polymerization was determined as previously described.¹⁴

Polymer Cleavage from Conjugates

Polymers were cleaved from the surface of CT using acid hydrolysis as previously described (Cummings et al. Biomaterials 34, 7437-7443 (2013)). Briefly, CT-conjugates (15 mg/mL) were dissolved in 6 N HCl and incubated at 110° C. under vacuum for 24 hours. Cleaved polymers were purified from CT by dialysis (MWCO 1 kDa) for 24 hours against deionized water, then lyophilized until a powder.

Molecular Weight and Dispersity of Cleaved Polymer

Gel permeation chromatography (GPC) was used to determine number (Mₙ) and weight average (M_{w}) molecular weights and polydispersity index (M_{w}/Mₙ) of cleaved polymer. GPC was performed on a Waters 2695 Series with a data processor and a refractive index (RI) detector.

Conjugate Hydrodynamic Diameter

CT-conjugates, native CT, and CTBr (0.5-1.0 mg/mL) were prepared in 100 mM sodium phosphate buffer (pH 8) and filtered using a 0.22 μM cellulose acetate syringe filter. Hydrodynamic diameter was measured using Particulate Systems NanoPlus (Micromeritics) dynamic light scattering at 25° C. with 25 accumulations in triplicate. Reported values are number distribution intensities.

Conjugate Zeta Potential

CT-conjugates, native CT, and CTBr (0.5 mg/mL) were prepared in 50 mM sodium phosphate buffer (pH 8) and filtered using a 0.22 μM cellulose acetate syringe filter. Zeta potential was measured using Particulate Systems NanoPlus (Micromeritics) at 25° C. with 5 cell positions.

Michaelis-Menten Kinetics

N-succinyl-Ala-Ala-Pro-Phe p-nitroanilide (Suc-AAPF-pNA) was used as a substrate for CT hydrolysis. Substrate (0-20 mg/mL in DMSO, 30 μL) was added to a 1.5 mL cuvette with 100 mM sodium phosphate buffer (pH 4, 6, 8, or 10). Native CT or CT-conjugates (0.1 mg/mL protein, 4 μM, 10 μL) was added to the cuvette with substrate and buffer. The initial substrate hydrolysis rate was measured by recording the increase in absorbance at 412 nm over the first 90 seconds after mixing using a Lambda 2 Perkin Elmer ultraviolet-visible spectrometer equipped with a temperature-controlled cell holder at 37° C. Michaelis-Menten parameters were determined using nonlinear curve fitting of initial hydrolysis rate versus substrate concentration in GraphPad. Kinetics were measured in triplicate.

Residual Activity Kinetics

CT-conjugates, native CT, and CTBr (1 mg/mL, 40 μM protein) were dissolved in 100 mM sodium phosphate buffer (pH 8). Samples were then diluted in triplicate to 4 μM using either 167 mM HCl (pH 1) or 10 mM NaOH (pH 12) and incubated in a circulating water bath at 37° C. Aliquots of 10 μL were removed at specific time points over 60 min and residual activity was measured in 100 mM sodium phosphate buffer (pH 8, 960 μL) using Suc-AAPF-pNA as a substrate (6 mg/mL, 30 μL, 288 μM in DMSO). Initial hydrolysis rate was determined by measuring the increase in absorbance at 412 nm over 40 seconds and data was normalized to its optimal activity at time 0. For disrupting electrostatic and hydrophobic interactions, either 1.0 M NaCl or 10 v/v % DMSO was added to the cuvette during incubation at pH 1. This was performed for each of the short length conjugates.

Tryptophan Fluorescence Refolding

CT-conjugates, native CT, and CTBr (1 mg/mL, 40 μM protein) were dissolved in 100 mM sodium phosphate buffer (pH 8). Samples were diluted (0.1 mg/mL, 4 μM) using either 167 mM HCl (pH 1) or 10 mM NaOH (pH 12) and incubated at 37° C. using a circulating water bath. After 40 min incubation, samples were diluted back to pH 8 (0.01 mg/mL, 0.4 μM) using 100 mM sodium phosphate (pH 8) into a 96 well plate in triplicate. The fluorescence intensity was measured by excitation at 270 nm and emission at 330 nm and 350 nm. The ratio of the emitted fluorescence intensity was calculated (350 nm/330 nm) and compared to the sample's original fluorescence intensity at time 0 (no incubation at pH 1 or 12). Percent change was calculated to determine refolding ability. Fluorescence was measured using a BioTek Synergy H1 Plate Reader at 37° C.

Tryptophan fluorescence kinetic unfolding CT-conjugates, native CT, and CTBr (1 mg/mL, 40 μM protein) were dissolved in 100 mM sodium phosphate buffer (pH 8). Samples were diluted to 0.1 mg/mL (4 μM protein) in a 96 well plate in triplicate using either 167 mM HCl (pH 1) or 10 mM NaOH (pH 12) (e.g. 30 μL sample and 270 μL of pH 1 or pH 12 solution). Fluorescence intensity was measured every 2 minutes over 40 minutes (excitation at 270 nm, emission at 330 nm and 350 nm). The ratio of emission (350 nm/330 nm) was plotted over time with time 0 as the fluorescence intensity of the sample at pH 8 (no incubation in pH 1 or pH 12). The temperature was held constant at 37° C. for 40 minutes and measurements were made using a BioTek Synergy H1 Plate Reader.

### Example 1. Design of Stomach Acid-Stable and Mucin Binding-Protein-Polymer Conjugates Comprising Chymotrypsin

Unmodified chymotrypsin is active from pH 5-10, with its pH optimum at pH 8 (see, e.g., al-Ajlan and Bailey Arch. Biochem. Biophys. 1997, 348, 363; and Castillo-Yanez et al. Food Chem. 2009, 112, 634). Interestingly, a cationic enzyme-polyamine conjugate was shown to be hyperactive at a wide range of pH (Kurinomaru et al. J. Mol. Catal. B: Enzym. 2015, 115, 135). Additionally, polymer-encapsulated, protein engineered, or polymer conjugated enzymes exhibit some stability under harsh digestive tract conditions (see, e.g., Xenos et al. Eur. J. Drug Metab. Ph. 1998, 23, 350; Rodriguez et al. Arch. Biochem. Biophys. 2000, 382, 105; Abian et al. Appl. Environ. Microbiol. 2004, 70, 1249; Qi et al. Animal 2015, 9, 1481; and Turner et al. Biotechnol Lett 2011, 33, 617. Chymotrypsin has a close to net-neutral surface charge and as a result native chymotrypsin is not mucoadhesive. Therefore, it was hypothesized that polymer-based protein engineering of chymotrypsin with charged polymers could generate enzyme variants that were mucin-binding and stable at extremes of acidic pH.

To further investigate the activity and mucin-binding of charged chymotrypsin-polymer conjugates, we grew neutral, zwitterionic, and charged polymers directly from the surface of chymotrypsin using atom-transfer radical polymerization. The polymers, poly(carboxybetaine acrylamide) (pCBAm(+/−)), poly(oligoethylene glycol methacrylate) (pOEGMA), poly(quaternary ammonium methacrylate) (pQA(+)), and poly(sulfonate methacrylate) (pSMA(−)), were chosen to incorporate charged moieties (sulfonate anion, ammonium cation) generally considered to be kosmotropes (order-making/stabilizing) in the Hofmeister series (see Zhang et al. Curr. Opin. Chem. Biol. 2006, 10, 658; and Baldwin Biophys. J. 1996, 71, 2056). Both positively and negatively charged polymers have been shown to possess mucoadhesive properties as a result of binding to the strongly hydrophilic glycosylated polymers that cover mucin proteins (see, e.g., Yin et al. Biomaterials 2009, 30, 5691; and Park and Robinson Pharm. Res. 1987, 4, 457). While uncharged and likely not mucoadhesive, pOEGMA has been shown to improve protein stability to different stressors such as temperature, protease degradation, and lyophilization (see, e.g., Rodriguez-Martinez et al. Biotechnol Lett 2009, 31, 883; Werle and Bernkop-Schnürch Amino Acids 2006, 30, 351; and Wang Int. J. Appl. Pharm. 2000, 203, 1). It was hypothesized that each of the conjugates would have an impact on enzyme stability at low pH while each polymer would have a distinct impact on mucin-binding of the chymotrypsin-polymer conjugates. In this first study, our goal was to elucidate the relationship between polymer physicochemical properties and the activity, stability, and mucin binding of the chymotrypsin-polymer conjugates.

### CT Conjugate Synthesis and Polymer Characterization

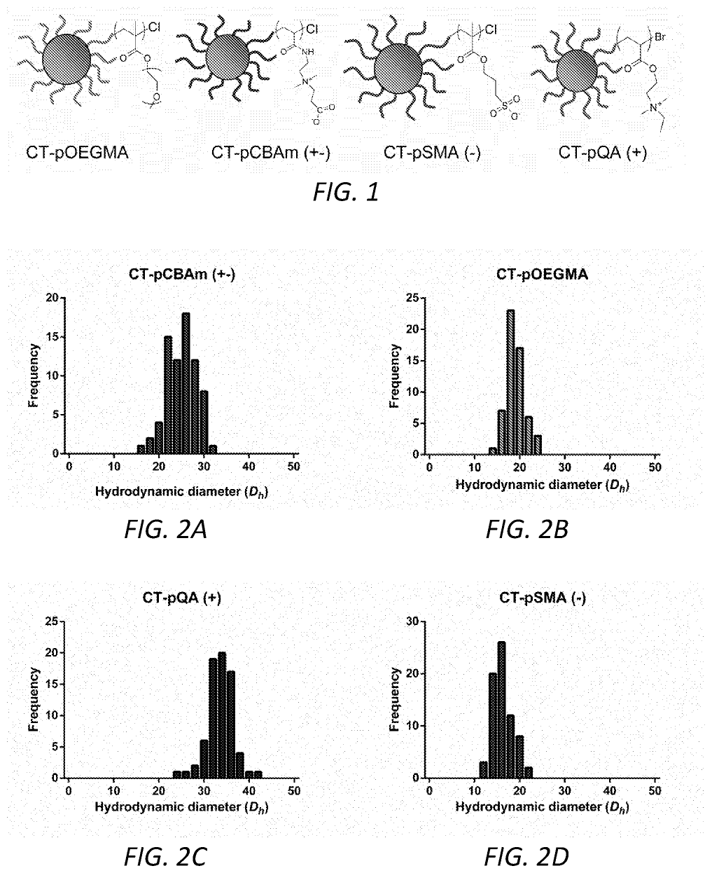

To determine the relationship between polymer physicochemical properties and enzyme activity, stability and mucin binding, four chymotrypsin-polymer biohybrid conjugates were designed and synthesized. pCBAm (+/−) was zwitterionic with a net neutral charge, pOEGMA was uncharged and neutral, pQA(+) was positively charged, and pSMA(−) was negatively charged. The polymers were grown directly from the surface of chymotrypsin using atom-transfer radical polymerization (ATRP) as described above. An idealistic representation of the final protein-polymer conjugates is shown in FIG. 1. The specific conditions for the synthesis of the enzyme-polymer conjugates, as listed in Table 1, were selected in order to optimize the polymerization with each of the monomers used.

Polymers were grown from 12 ATRP initiator sites (as calculated by MALDI-TOF-MS) covalently attached to surface accessible lysine residues using NHS-ester/amine chemistry. Successful polymerization from chymotrypsin was confirmed using dynamic light scattering (DLS), and each chymotrypsin-polymer conjugate had a similar increase in hydrodynamic diameter (Dₕ) compared to native chymotrypsin (5.7±2 nm) (FIGS. 2A-2D).

To characterize polymers grown from chymotrypsin, the polymers were cleaved from the surface of chymotrypsin using acid hydrolysis in 6 N HCl. Polymer molar mass was calculated using size exclusion GPC, and polymer molar mass values correlated well with hydrodynamic diameters measured by DLS. (Table 1)

TABLE 1
Molecular weight and hydrodynamic
diameter of chymotrypsin conjugates

| | | Cleaved | | | |
|---|---|---|---|---|---|
| | | Polymer | | Conjugate | |
| | | | PDI | Molar | Size |
| | | Mₙ | (M_{w}/ | Mass | (Dₕ) |
| | Cu/Ligand Pair | (kDa) | Mₙ) | (kDa) | [nm] |
| CT-pCBAm | CuCl:Me6TREN | 30.7 | 1.90 | 393 | 26.3 ± 3.2 |
| (+/−) | | | | | |
| CT-pOEGMA | CuCl:CuCl2:bpy | 11.6 | 1.46 | 165 | 20.1 ± 2.0 |
| CT-pQA (+) | CuBr:HMTETA | 19.1 | 2.10 | 254 | 34.5 ± 2.9 |
| CT-pSMA(−) | CuCl:CuCl2:bpy | 9.6 | 1.43 | 140 | 17.2 ± 2.2 |

### In Vitro Mucin-Binding of ATRP-Synthesized Free Polymers

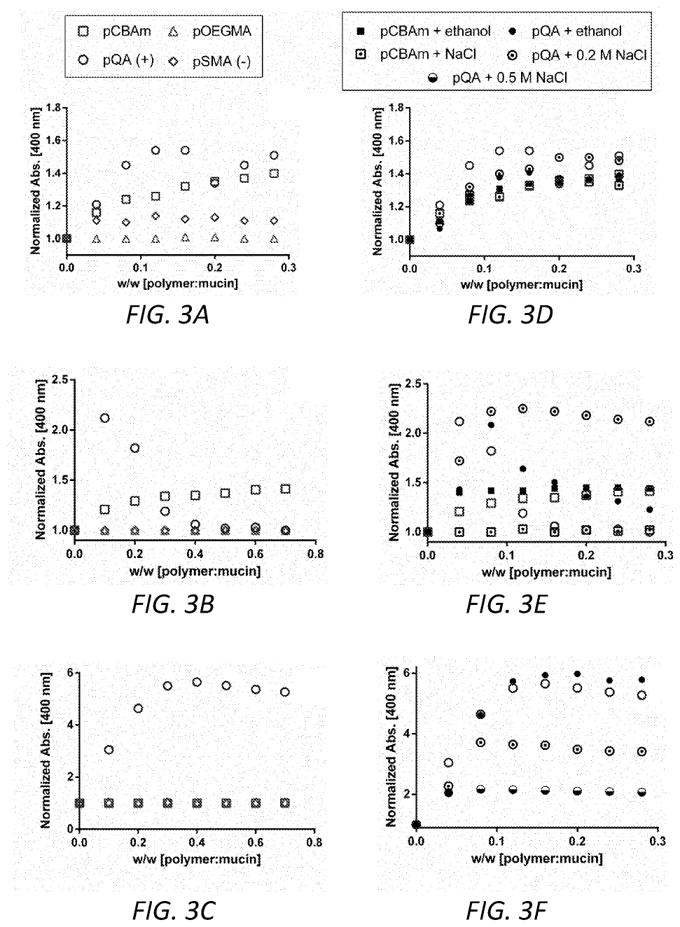

Exogenous enzymes modified with mucin-binding molecules could exhibit increased residence time in the GI tract. In order to examine the in vitro pH-dependence of mucin-binding properties of each of the polymers that were grown from chymotrypsin, the turbidities of mucin protein solutions (at 37° C.) with increasing free polymer content at pH 1 (167 mM HCl), pH 4.5 (50 mM ammonium acetate buffer), and pH 8.0 (50 mM sodium phosphate buffer) were measured. In this assay, an increase in the turbidity of mucin colloidal suspensions is driven by mucoadhesive polymer-mediated crosslinking of mucin.⁵¹ The positively charged pQA (+) polymer exhibited significant mucin binding across the range of pH values tested. The degree of mucin binding of the zwitterionic polymer, pCBAm, was pH-dependent whereas the neutral polymer, pOEGMA, was not mucoadhesive at any pH. The negatively charged polymer, pSMA (−), was also non-mucin binding across the range of pH tested (FIGS. 3A-3C).

Mucoadhesion in vivo results from a balance of electrostatic interactions, hydrogen bonding, and hydrophobic interactions with mucin (Smart Adv. Drug Delivery Rev. 2005, 57, 1556). Sialic acid, a major component in mucin, is a polysaccharide with carboxylic acid functionality giving mucin a net negative charge at neutral pH (see, e.g., Leach Nature 1963, 199, 486). Since the positively charged polymer, pQA (+), increased the turbidity of mucin suspensions from pH 1-8, it was hypothesized that electrostatic interactions were the main driving force for observed pQA (+) mucoadhesion. To test this hypothesis further, the ionic strength of the mucin suspension with the addition of sodium chloride (NaCl) was increased. Separately, to rule out hydrophobic interactions as the mucoadhesive driving force, the hydrophobicity of the solution with the addition of ethanol was increased. An electrostatic attraction-mediated mucin binding was expected to be diminished by the salt-mediated increase in ionic strength, whereas a hydrophobic interaction-mediated binding would be diminished by ethanol. Both pCBAm and pQA (+) mucin suspension turbidities were unaffected by the addition of ethanol, but dependent on ionic strength (FIGS. 3D-3F). The addition of NaCl affected polymer mucin binding for both pQA (+) and pCBAm by either decreasing the absolute turbidity or the shifting of turbidity plateau to higher polymer:mucin ratios. From these results, it was clear that the mucoadhesion of pQA (+) (at every pH) and pCBAm (at low pH) was due to electrostatic attraction of the positively charged polymers with the negatively charged mucin. To further confirm this hypothesis, the zeta potentials of the free polymers and mucin were measured at each pH (Table 2). The zeta potentials of each of the polymers correlated well with electrostatic interactions being responsible for the behavior seen in the in vitro mucoadhesion experiments. The uncharged free polymer, pOEGMA, did not show mucoadhesive properties at any of the pH values tested which was not surprising given the existing literature (Fuhrmann et al. Nat. Chem. 2013, 5, 582). Predictably, pSMA (−) was also not mucoadhesive, likely due to electrostatic repulsion of the negative charge in the polymer and negatively charged mucin.

TABLE 2
Zeta potential (ζ) measurements of free polymer in mucoadhesive relevant solutions

| | Zeta potential (ζ) [mV] | | | | |
|---|---|---|---|---|---|
| | Mucin | pCBAm | pOEGMA | pQA (+) | pSMA (−) |
| 50 mM Citric acid (pH 2.3) | 0.7 ± 0.4 | 15 ± 6 | 1.9 ± 0.6 | 34 ± 10 | −22 ± 3.6 |
| 50 mM (NH₄⁺) acetate (pH 4.5) | −3.6 ± 0.5 | 0.3 ± 0.4 | 2.9 ± 2.3 | 29 ± 5.9 | −25 ± 2.6 |
| 50 mM NaPhos (pH 8.0) | −7.1 ± 0.7 | −2.0 ± 1.8 | 1.2 ± 4.5 | 7.8 ± 4.0 | −22 ± 5.1 |

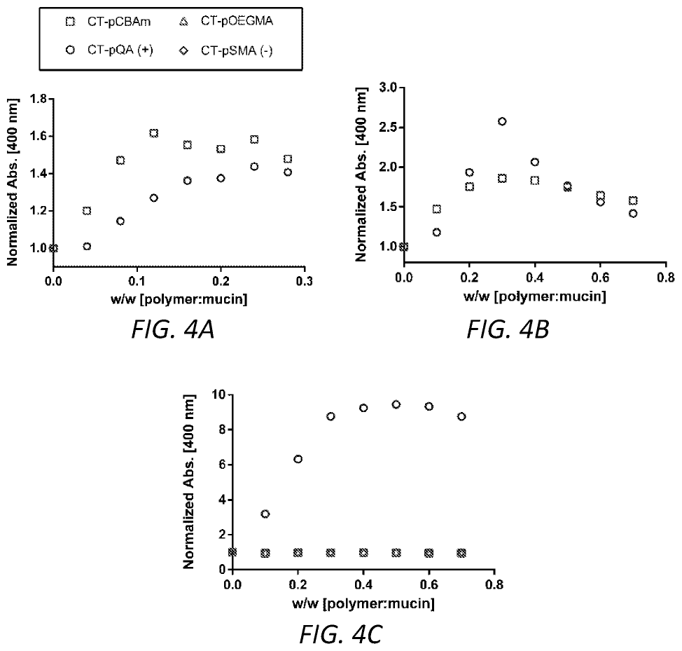

While it was clear that pQA (+) and pCBAm did indeed have mucoadhesive properties, several unexpected and interesting trends resulted for these polymers. The pH responsive behavior of pCBAm was likely due to the ionization state of the carboxylic acid in the polymer at each of the test pH values. In highly acidic conditions (pH 1), protonation occurred in pCBAm, resulting in a net positive charge. However, at pH 4.5 and pH 8, no mucoadhesion was observed for pCBAm due to deprotonation and a net neutral charge. While pQA (+) was mucoadhesive at each pH tested, the normalized absorbance values after incubation for pQA (+) polymers were much higher at pH 8 compared to both pH 1 and pH 4.5. This result was likely due to the reduced number of negatively charged crosslinking sites in mucin at pH 1. As described earlier, carboxylic acid functionality is responsible for the negative charge in mucin, so it was not surprising to see less of a crosslinking effect at low pH values. Interestingly, at pH 4.5, pQA (+) normalized turbidity initially increased before returning to baseline levels at 0.3 w/w ratios. At this pH, it was likely that, at higher ratios of pQA (+), the polymer fully encapsulated mucin particles rather than crosslink between particles, resulting in solubilization and lower turbidity. These results led to the hypothesis that the CT-pQA (+) conjugate would also be mucin binding. Indeed, CT-pQA (+) conjugates were mucin binding at pH 1.0, pH 4.5, and pH 8 (FIGS. 4A-4C). At pH 8, neither CT-pCBAm (+/−), CT-pOEGMA, nor CT-pSMA (−) showed mucin binding behavior. As in the case of the free polymer, CT-pCBAm was mucin-binding at pH 1.0 and pH 4.5, which may be due to the protonation of the carboxylic acid at low pH and a resulting net positive charge. Mucin-binding properties of CT-pOEGMA and CT-pSMA (−) conjugates were not determined at low pH due to protein structural unfolding induced by the polymers.

### Impact of Polymer Charge State on the Activity of Chymotrypsin-Polymer Conjugates

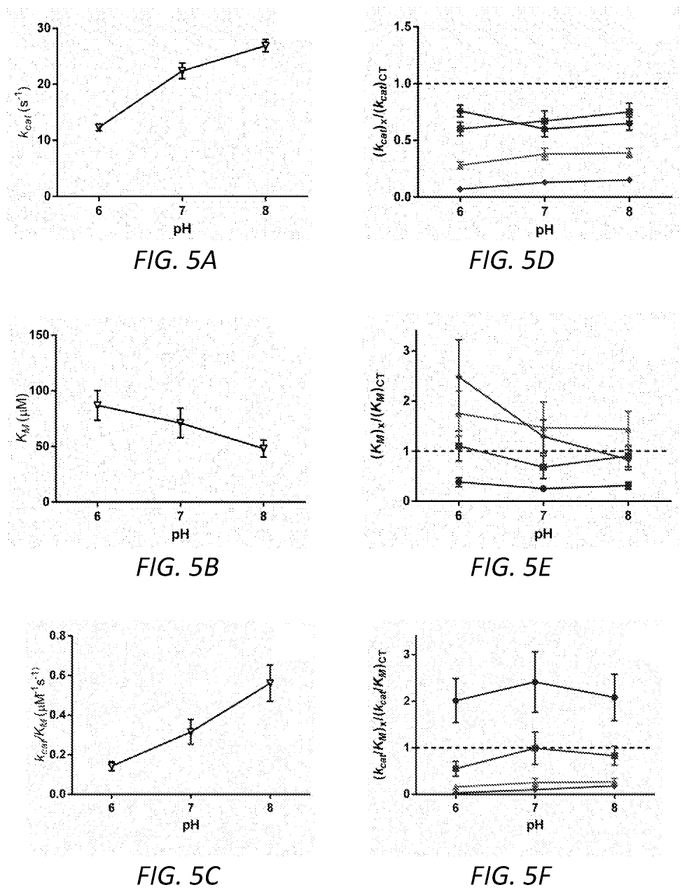

Kinetic rate (k_{cat}) and substrate binding (K_{M}) constants for the ATRP-synthesized CT-polymer conjugates were determined using a short peptide substrate, N-succinyl-Ala-Ala-Pro-Phe-p-nitroanilide, in 100 mM sodium phosphate buffer (pH 6-8) at 37° C. Conjugate activity was dependent on the properties of the covalently attached polymer in the protein-polymer conjugate (FIGS. 5A-5F). Relative turnover number (k_{cat}) values for each conjugate were independent of pH, and all decreased compared to native chymotrypsin. CT-pSMA (−) and CT-pOEGMA activity values were both less than half that of native chymotrypsin, while CT-pQA (+) and CT-pCBAm maintained approximately 70% of native chymotrypsin activity after modification. A reduction in k_{cat} has often been observed for enzyme-polymer conjugates with the prevailing hypothesis being that the polymer causes a structural stiffening of the enzyme, though definitive mechanisms have never been determined (see, e.g., Rodriguez-Martinez Biotechnol. Bioeng. 2008, 101, 1142). Previously, a decrease in relative k_{cat} values for CT-pSBAm-b-pNIPAM conjugates was observed (Cummings et al. Biomacromolecules 2014, 15, 763). The more significant decrease in activity for CT-pSMA (−) and CT-pOEGMA could have been due to interactions between those specific polymers and chymotrypsin. A large increase in substrate affinity was observed for CT-pQA (+) conjugates, as evidenced by the decrease in K_{M} values. CT-pOEGMA had decreased substrate affinity relative to the native unmodified enzyme, whereas the CT-pCBAm had similar substrate affinity. The K_{M} values for both conjugates were not pH-dependent from pH 6-8. Interestingly, the CT-pSMA (−) conjugate relative K_{M} values were pH-dependent and substrate affinity was decreased at pH 6. The effect of polymer on K_{M} of the CT-pSMA (−) and CT-pQA (+) conjugates for the substrate were most likely due to electrostatic repulsion and attraction, respectively, between the polymer coat around the enzyme and the negatively charged substrate. As a negatively charged substrate molecule, the affinity of the substrate for chymotrypsin has previously been shown to be affected by polymer conjugation (Riccardi et al. Bioconjugate Chem. 2014, 25, 1501). The significant K_{M} effects drove increased CT-pQA (+) productivity (k_{cat}/K_{M}) values relative to the native unmodified chymotrypsin. Both CT-pSMA (−) and CT-pOEGMA productivities were lower than native chymotrypsin at each pH, and CT-pCBAm conjugates showed similar productivity to native chymotrypsin at each pH. Importantly, activity for each of the chymotrypsin-polymer conjugates was measured against a four peptide long substrate and activity of these conjugates could be different if activity was tested with a larger protein substrate (Lucius et al. Biomacromolecules 2016, 17, 1123). In vivo, chymotrypsin-polymer conjugates would work most effectively in the intestines, because neutral pH is optimum and foodstuff proteins would already be broken down to peptides by pepsin and acid in the stomach.

### Impact of Polymer Charge State on the Stability of Chymotrypsin-Polymer Conjugates at pH 1.0

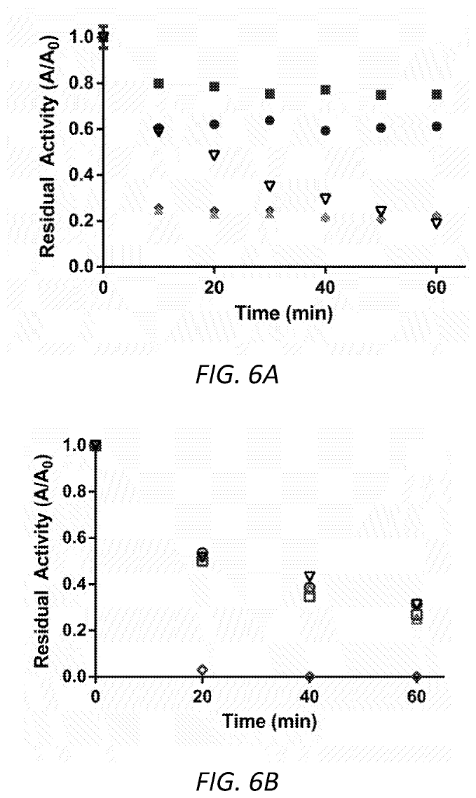

The rate of irreversible inactivation of the CT conjugates at low pH was determined by incubating conjugates in 167 mM HCl at 37° C. and measuring residual activity at specified time points. (FIG. 6A) The stability of CT conjugates in acid was dependent on the polymer attached to chymotrypsin. Both CT-pCBAm (+, at low pH) and CT-pQA (+) conjugates were more stable than native chymotrypsin, with stability profiles similar to what was observed previously for CT-pSBAm and CT-pQA conjugates (see Cummings et al. Biomacromolecules 2014, 15, 763). CT-pOEGMA and CT-pSMA (−) conjugates both lost activity more rapidly than native chymotrypsin, showing that both pOEGMA and pSMA (−) had a destabilizing effect on chymotrypsin. The addition of free pQA (+) or pCBAm (+) polymers to native unmodified chymotrypsin did not stabilize the enzyme from acid-mediated irreversible inactivation, indicating that the covalent attachment of polymers was required for increased stability. (FIG. 6B)

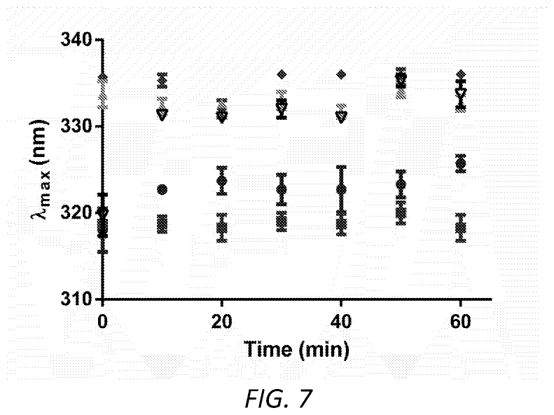

The addition of pSMA (−) free polymers to unmodified chymotrypsin actually decreased activity compared to native chymotrypsin, confirming the destabilization effect of pSMA (−) towards chymotrypsin. Conversely, while CT-pOEGMA conjugates showed similar low stability to CT-pSMA (−) conjugates when incubated in 167 mM HCl, native chymotrypsin incubated with free pOEGMA had a similar stability profile to native chymotrypsin, chymotrypsin with pQA (+), and chymotrypsin with pCBAm (+). These data help contribute to our understanding of a proposed mechanism by which the covalently attached polymers either stabilize or destabilize enzymes. In order to develop that mechanism further, we examined the effect of ATRP-grown polymers on the structural integrity of the enzyme by following intrinsic tryptophan fluorescence for each of the conjugates after exposure to low pH. (FIG. 7)

Intrinsic tryptophan fluorescence is a recognized sentinel for changes in protein tertiary structure. Protein unfolding leads to increases in the maximum wavelength of fluorescence emission (μₘₐₓ). Each CT conjugate was incubated at pH 1.0 in 167 mM HCl and fluorescence emission spectrum was measured from 300-400 nm after excitation at 270 nm. Native chymotrypsin λₘₐₓ increased from 320 nm to 334 nm over 60 minutes. This result was expected as native chymotrypsin has been previously shown to irreversibly unfold at low pH (Kijima et al. Enzyme Microb. Technol. 1996, 18, 2; and Desie et al. Biochemistry 1986, 25, 8301). Not surprisingly, both CT-pSMA (−) and CT-pOEGMA also had increased lambda max values compared to native chymotrypsin starting at t=0 min. Conversely, CT-pQA (+) had increased λₘₐₓ values over the course of the experiment, but the increase was not as large in magnitude as native chymotrypsin, indicating significantly less extensive irreversible unfolding for CT-pQA (+) at low pH. CT-pCBAm (+ at pH 1.0) conjugates showed the least amount of unfolding during this experiment as the λₘₐₓ remained almost unchanged during the course of the experiment. From this experiment, it was clear that the loss in activity for both CT-pOEGMA and CT-pSMA (−) conjugates was due to unfolding and not enzyme autolysis. The results of the intrinsic fluorescence experiments correlated well with residual activity measurements, where both CT-pQA (+) and CT-pCBAm (+ low pH) were more stable than native chymotrypsin. CT-pCBAm (+ at low pH) had higher activity during the course of the experiment. In addition, both CT-pSMA (−) and CT-pOEGMA lost activity quickly during residual activity experiments, and this reduced activity coincided with a large increase in λₘₐₓ values during intrinsic fluorescence experiments.

### Mechanism of Stabilization of Enzymes by Conjugated “Grown-From” Polymers

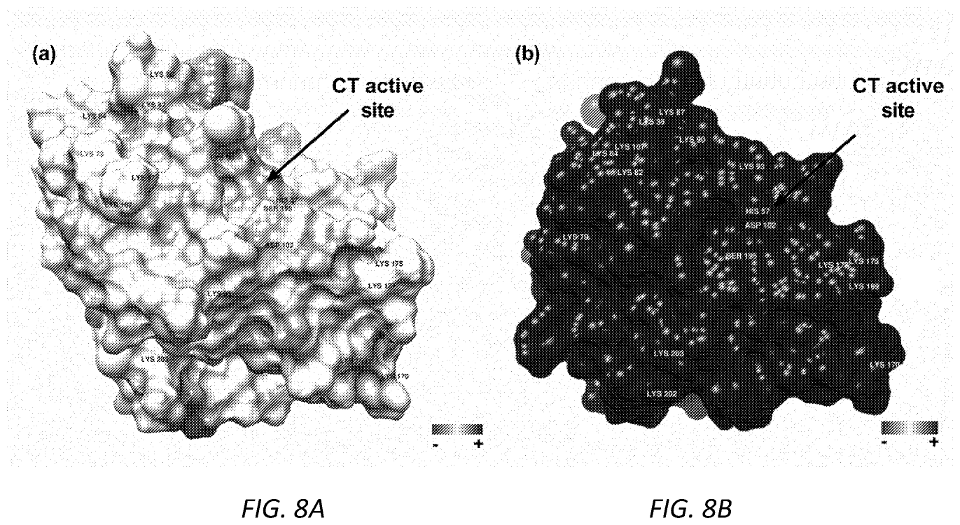

The data generated for the impact of polymer charge on both activity and tertiary structure align well with data generated previously when determining the impact of anionic nanoparticles on the activity and unfolding of chymotrypsin (Fischer P. Natl. Acad. Sci. U.S.A. 2002, 99, 5018). In that work, it was hypothesized that the anionic nanoparticles selectively associated with a cationic core of amino acid residues around the chymotrypsin active site. In addition, the authors hypothesized the hydrophobic nature of the nanoparticles also led to detrimental effects on chymotrypsin stability and activity due to a region of hydrophobic residues also near the active site. Of the destabilizing polymers used in this study, one was negatively charged (pSMA) (−) and the other was amphiphilic (pOEGMA). While the inhibition and destabilizing properties of negatively charged molecules seemed to be conserved in this study, the surface charge of initiator modified chymotrypsin (CT-Br) must be considered rather than native chymotrypsin. Indeed, a large amount of positively charged surface area was lost when coupling the ATRP initiator onto surface lysine residues, which bear a positive charge in native chymotrypsin at neutral pH. We therefore performed a 10 ns molecular dynamics simulation of CT-Br in water to obtain a representative structure of the initiator complex. Ionization states of CT-Br at pH 1 and 7 were predicted using PROPKA 2.0 after which surface charge analysis was possible by calculating electrostatic potentials according to Coulomb's Law. At pH 7 molecular dynamic simulations showed that the region of positive charge near the active site remains in the initiated enzyme and, thus, this region could exhibit specific interactions with charged conjugated polymers. (FIG. 8A) In addition, at pH 1, where the stability experiments were conducted for this study, CT-Br bore a global positive surface charge. (FIG. 8B)

Other studies have observed that denaturing osmolytes (urea, guanidine hydrochloride) preferentially accumulated at the protein surface, whereas stabilizing osmolytes (TMAO, betaine) were preferentially excluded from the surface (see, e.g., Street et al. P. Natl. Acad. Sci. U.S.A. 2006, 103, 13997). This preferential accumulation or exclusion of osmolytes was due to either specific interactions of the osmolytes with the protein or a global alteration of water structure (see Bennion and Daggett P. Natl. Acad. Sci. U.S.A. 2004, 101, 6433). In any case, stabilizing osmolytes resulted in a stronger hydration layer which strengthened protein structural stability, and denaturing osmolytes displaced water molecules in the hydration layer causing lower stability (Timasheff P. Natl. Acad. Sci. U.S.A. 2002, 99, 9721). Destabilizing osmolytes interact with the protein by reducing the thermodynamic penalty for exposing hydrophobic residues usually confined to the protein core. Conversely, stabilizing osmolytes increase the thermodynamic barrier for proteins to transfer to the unfolded from the folded state.

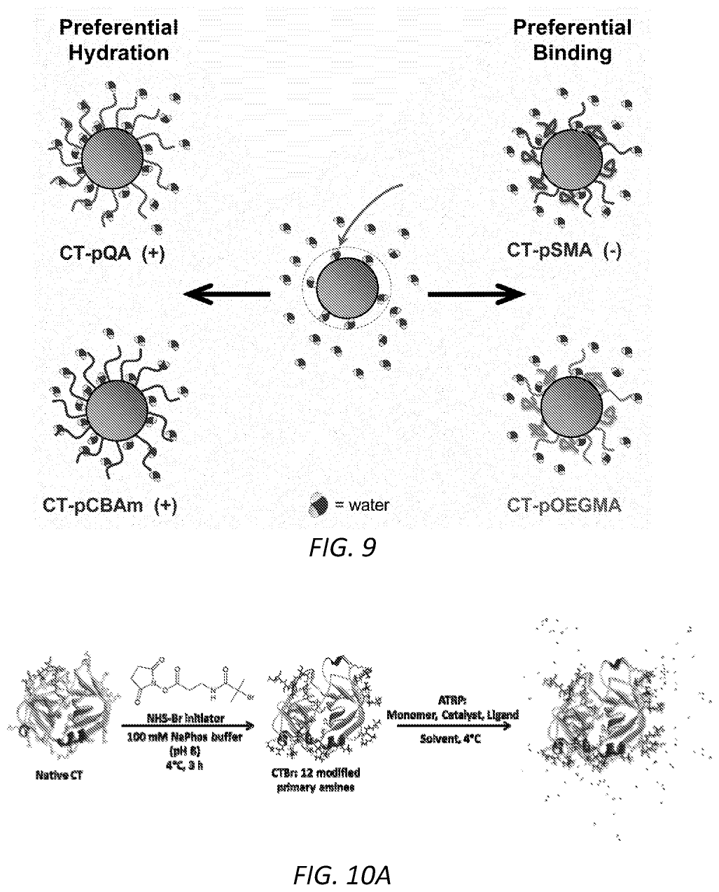

Specifically for protein-polymer conjugates, other reports indicated that proteins were stabilized after polymer conjugation due to favorable interactions between the protein and polymer (see Lucius et al. Biomacromolecules 2016, 17, 1123; and Mancini, et al. J. Am. Chem. Soc. 2012, 134, 8474). In these examples, the inventors hypothesized that the polymer non-covalently bound with the protein and stabilized the protein through a mechanism similar to cross-linking. Separately, Price et al. has extensively examined the effect of PEGylation on protein stabilization (see Lawrence and Price Curr. Opin. Chem. Biol. 2016, 34, 88). They found that PEGylation can stabilize proteins both by PEG extension into the solution or PEG interaction with the protein surface (Chao J. Phys. Chem. B 2014, 118, 8388). Importantly, they determined that PEGylation can be stabilizing or destabilizing in the WW domain of human protein pin 1 depending on the location of attachment, and that conjugation strategy and length of PEG both heavily influence conformational stability (see Lawrence et al. J. Am. Chem. Soc. 2014, 136, 17547; Lawrence et al. ACS Chem. Biol. 2016, 11(7), 1805; and Pandey et al. Bioconjugate Chem. 2013, 24, 796). It was hypothesized that conjugates grown from pQA (+) and pCBAM (+) stabilized chymotrypsin to low pH structural unfolding by preferential exclusion of the polymer from chymotrypsin surface. (FIG. 9) Since polymer interactions with chymotrypsin were thermodynamically unfavorable, the water hydration layer was strengthened, and thereby increased structural stability. It was also hypothesized that pSMA (−) and pOEGMA destabilized chymotrypsin through a preferential interaction between the polymer and the protein surface. The differing stabilization profiles for native chymotrypsin incubated with free pSMA (−) and pOEGMA polymer indicated that the mechanism of destabilization may be different for these two polymers. Growing polymers from the surface of a protein may be stabilizing if the polymer and protein surface are designed to not interact strongly with each other (and vice versa). In addition, to see a stabilizing effect, the solvent environment around the protein must not reduce the penalty of exposed hydrophobic residues. Conversely, destabilizing polymers manipulate the solvent environment to reduce the penalty of exposed hydrophobic residues. Naturally, electrostatic forces, hydrogen bonding, and hydrophobic interactions all drive protein surface-“grown from” polymer interactions. The data with CT-pOEGMA demonstrate that minimizing hydrophobic interactions between the polymer and the protein surface was important, but pSMA (−) destabilization indicates more than just hydrophobic interactions are important for conformational stability. One of the most exciting aspects of growing polymers from the surface of proteins is the ability to target and tune the properties of the polymer.

Here, four different chymotrypsin-polymer conjugates were synthesized using surface initiated ATRP polymer-based protein engineering. The four conjugates, CT-pCBAm (+/−), CT-pOEGMA, CT-pQA (+), and CT-pSMA (−), each had different mucoadhesive, bioactivity, and stability profiles. CT-pQA (+) and CT-pCBAm (+/−) conjugates were mucoadhesive and maintained bioactivity at all pH values tested, whereas CT-pOEGMA and CT-pSMA (−) were not mucoadhesive and had reduced activity. Most importantly, CT-pQA (+) and CT-pCBAm (+/) conjugates stabilized chymotrypsin, whereas CT-pSMA (−) and CT-pOEGMA destabilized the enzyme to the low pH structural denaturation. It was hypothesized that the different stabilization properties were due to preferential accumulation of the destabilizing polymers and preferential exclusion of the stabilizing polymers at the enzyme surface. This accumulation and exclusion likely influenced the integrity of the surface hydration layer which led to structural destabilization and stabilization, respectively. Due to their increased stability and maintained activity, CT-pCBAm (+/−) and CT-pQA (+) would be better candidates than CT-pOEGMA or CT-pSMA (−) as an exogenous chymotrypsin enzyme replacement therapy.

### Example 2. Structure Function Relationships of Protein-Polymer Conjugates Comprising Chymotrypsin

Working at the interface of synthetic chemistry and biology has created interest in protein-polymer conjugates in industries as diverse as therapeutics, diagnostics, sensing, synthetic synthesis, food and cosmetics, and biotechnology (see, e.g., Wu et al. Biomater. Sci. 3, 214-30 (2015); Veronese and Pasut Drug Discov. Today 10, 1451-1458 (2005); Heredia et al. J. Am. Chem. Soc. 127, 16955-60 (2005); Cobo et al. Nat. Mater. 14, 143-159 (2014); Hills Eur. J. Lipid Sci. Technol. 105, 601-607 (2003); Bi et al. J. Agric. Food Chem. 63, 1558-1561 (2015); and Choi et al. Biotechnol. Adv. 33, 1443-1454 (2015)). There has also been increasing interest in whether polymer conjugation can also enhance enzyme activity and stability in non-native environments, but the absence of a fundamental understanding of how polymers impart that benefit limits the ability to rationally design bioconjugates with polymer-based protein engineering (Klibanov Trends. in Biochem. 14, 141-144 (1989); and Klibanov Nature 409, 241-246 (2001)). The first reported protein-polymer conjugate was synthesized in 1977 by covalently attaching poly(ethylene glycol) (PEG) to bovine serum albumin showing enhanced properties over the native protein (see Abuchowski et al. J. Control. Rel. 252, 3578-3581 (1977); and Abuchowski et al. J. Biol. Chem. 252, 3582-3586 (1976)). More recently, efforts have focused on fully exploiting the properties of polymers to create functional and responsive “smart conjugates” (see, e.g., Heredia et al. J. Am. Chem. Soc. 127, 16955-16960 (2005); Cobo et al. Nat. Mater. 14, 143-159 (2014); Kulkarni et al. Biomacromolecules 7, 2736-2741 (2006); Grover and Maynard Curr. Opin. Chem. Biol. 14, 818-827 (2010); and Cummings et al. Biomaterials 34, 7437-7443 (2013)). Examples include temperature and pH responsive polymers to alter solubility and substrate affinity (see, e.g., Cobo et al. (2014); Cummings Biomaterials 34, 7437-7443 (2013); Cummings et al. Biomacromolecules 2014, 15(3): 763-71; Murata et al. Biomacromolecules 14, 1919-1926 (2013)). Now that polymer structure can be varied synthetically, it has become important to develop predictive rules, algorithms and models that can enhance the rationality of the field (Cummings et al. (2013); Cummings et al. (2014); Murata et al. (2013); Campbell et al. Biosens. Bioelectron. 86, 446-453 (2016); and Carmali et al. ACS Biomater. Sci. Eng., 2017, 3(9): 2086-97). There are now many tools, that fall into “grafting-to” and “grafting-from” strategies, through which protein-polymer conjugates can be synthesized (Wilson Macromol. Chem. Phys. 218, 1600595 (2017)). In the “grafting-from” approach, an initiator is first reacted with the surface of a protein, typically using surface accessible primary amines, and polymer chains are grown from the initiator sites monomer by monomer using controlled radical polymerization techniques (Grover and Maynard (2010); Lele et al. Biomacromolecules 6, 3380-3387 (2005); Cummings et al. Biomacromolecules 2017, 18(2): 576-586; Pelegri-O'Day and Maynard Acc. Chem. Res. 49, 1777-1785 (2016); Murata et al. Biomacromolecules 15, 2817-2823 (2014); and Averick et al. ACS Macro Lett. 1, 6-10 (2012)). “Grafting-from” is particularly well suited to higher modification density, control over polymer architecture (length and monomer type), and enhanced control over attachment site resulting in exceptionally uniform conjugates. Although a plethora of “grafted-from” protein-polymer conjugates synthesized using atom-transfer radical polymerization (ATRP) have been studied, a molecular understanding of how the conjugated polymer affects a protein's activity and stability has not been ascertained (Cummings et al. (2013); Cummings et al. (2014); Campbell et al.; Lele et al. (2005); and Cummings et al. (2017)).

PEG is undoubtedly the most commonly used polymer to synthesize and study conjugates, but due to an increased concern about its immunogenicity and lack of functionality, scientists are beginning to explore other polymer types as well (see, e.g., Schellekens et al. Pharm. Res. 30, 1729-1734 (2013)). Several reports have claimed increased protein stability upon polymer conjugation, however there are also contradictory results where conjugates behaved similar to their native protein. There are also conflicting results as to whether or not an increase in conjugated polymer molecular weight increases protein stability, and if it does, the explanation as to why is typically vague. Furthermore, for results that share a common theory, the mechanistic hypotheses for stabilization are often different. The most quoted theory (generalized) in the literature is that polymers preferentially interact with the protein surface. In some cases, the polymers form hydrogen bonds and hydrophobic interactions with the protein surface leading to stabilization. In other cases, the polymer provides a hydration layer around the protein surface that either reduces it propensity to aggregate or simply provides steric shielding from the denaturing environment. An alternative theory hypothesizes that polymers do not interact with the protein surface and maintain their flexibility in solution while still providing stabilizing effects. A table of these studies has been complied to more easily compare and contrast these hypotheses (Table 3). It still remains unclear what the stabilizing mechanism for conjugates is and whether or not it depends on polymer molecular weight, charge, and denaturing environment (temperature or chemical).

TABLE 3

| | | | | | | Stability | |
|---|---|---|---|---|---|---|---|
| | | | | | | increases | |
| | | Polymer | Polymer | Stability | Hypothesized Stabilizing | with polymer | |
| Protein | Polymer | Size | Density | Type | Mechanism | M_{w}? | Ref. |
| Chymotrypsin | PEG | 0.7, 2, 5 | 10-65% | Thermal | PEG increased protein | No | Rodriguez- |
| | | kDa | of amino | | thermal stability by | | Martinez et al. |
| | | | groups | | decreasing structural | | Biotechnol. |
| | | | | | dynamics because | | Bioeng. |
| | | | | | hydrophobic regions of | | 101, 1142-9 |
| | | | | | PEG bind the protein | | (2008). |
| | | | | | surface, exclude water, | | |
| | | | | | and make the protein | | |
| | | | | | more rigid. Stability | | |
| | | | | | increased with the | | |
| | | | | | density of polymer | | |
| | | | | | modification | | |
| | Spermidine | 0.25 kDa | 0-1 mM | Thermal | Bound spermidine | — | Farhadian |
| | (noncovalent) | | was | | interacts with the | | et al. Int. J. |
| | | | added | | protein through VDWs | | Biol. Macromol. |
| | | | | | and H-bonding leading to | | 92, 523-32 |
| | | | | | increased thermal | | (2016). |
| | | | | | stability | | |
| | pCBAm | 30.7 kDa | 80% of | Acid | Conjugate stability | — | Cummings |
| | pQA | 19.1 kDa | amino | | against acid was | | et al. |
| | pSMA | 9.6 kDa | groups | | increased due to | | Biomacromolecules |
| | pOEGMA | 11.6 kDa | | | extension of polymer | | acs. biomac. |
| | | | | | from the protein surface | | 6b01723 |
| | | | | | to minimize electrostatic | | (2017). |
| | | | | | interactions | | |
| Trypsin | PEG | 5 kDa | 80% of | Thermal | Thermal and detergent | — | Gaertner and |
| | | | amino | and | stability increased from | | Puigserver |
| | | | groups | Detergent | the formation of a highly | | Enzyme |
| | | | | | H-bonded structure | | Microb. Technol. |
| | | | | | around the enzyme | | 14, 150-5 |
| | | | | | | | (1992). |
| | Dextrin | Dextrin: 17 | 1 or 2 | Thermal | Conjugates showed | Yes | Treetharnm |
| | ST-HPMA | or 64 kDa | chains | and | increased thermal | | athurot et |
| | | ST-HPMA: | | Autolytic | stability and better | | al. Int. J. Pharm. |
| | | 12 kDa | | | stability to autolysis. | | 373, 68-76 |
| | | | | | Higher MW polymer | | (2009). |
| | | | | | enhanced protection | | |
| | | | | | from autolytic attack due | | |
| | | | | | to steric hindrance and | | |
| | | | | | H-bonding | | |
| Lysozyme | PEG | 5 kDa | 1 chain | Thermal | Thermal stability of | — | Nodake and |
| | | | | | conjugates increased due | | Yamasaki |
| | | | | | to H-bonding between | | Biosci. |
| | | | | | the ethylene oxide | | Biotechnol. |
| | | | | | groups and the protein | | 8451, |
| | | | | | | | (2000). |
| | Am | Low and | 90% of | Thermal | Conjugates, independent | Thermal: | Lucius et al. |
| | DMAm | High M_{w} | amino | and | of polymer charge, had | No | Biomacromolecules |
| | OEOA | Range: ~0.6-53 | groups | Chemical | decreased thermal | Chemical: | 17, 1123-1134 |
| | Am/PCMA | kDa | | | stability and higher | Yes | (2016). |
| | Am/AA | | | | molecular weight further | | |
| | Am/DMAEMA | | | | decreased thermal | | |
| | AGA | | | | stability. This was | | |
| | | | | | attributed to the larger | | |
| | | | | | polymers causing | | |
| | | | | | unfavorable folding | | |
| | | | | | entropy. | | |
| | | | | | Increased polymer | | |
| | | | | | molecular weight | | |
| | | | | | increased chemical | | |
| | | | | | stability. Ionic polymers | | |
| | | | | | improve stability by | | |
| | | | | | interacting with the | | |
| | | | | | protein surface in | | |
| | | | | | comparison to nonionic | | |
| | | | | | polymers of similar | | |
| | | | | | molecular weight. | | |
| | PEG | 2, 5, 10 | 1 or 2 | Thermal | Polymer conjugation did | No | Morgenster |
| | | kDa | chains | | not improve thermal | | net al. Int. J. |
| | | | | | stability | | Pharm. |
| | | | | | | | 519, 408-17 |
| | | | | | | | (2017). |
| Pyrophosphatase | pOEGMA | 4, 8, 12 | 1 chain | Thermal | High polymer molecular | Yes | Cao et al. |
| | pNIPAAm | kDa | | | weights increase thermal | | Polym. |
| | via host-guest | | | | stability. Polymer length | | Chem. |
| | interactions | | | | needs to be longer than | | 7, 5139-46 |
| | (non-covalent) | | | | the distance between | | (2016). |
| | | | | | the attachment site and | | |
| | | | | | active center. pOEGMA | | |
| | | | | | stabilized the protein at | | |
| | | | | | high temperatures by | | |
| | | | | | forming a hydration layer | | |
| | | | | | around the protein to | | |
| | | | | | reduce aggregation. | | |
| | pNIPAAm | M_{w} | 1 chain | Thermal | Thermal stability was | — | Yang et al. |
| | | conjugate: ~50 | | | increased due to the | | ACS Appl. |
| | | kDa | | | hydrophobic collapse of | | Mater. Interfaces |
| | | | | | pNIPAAm above its LCST. | | 8, 15967-74 |
| | | | | | This conformation | | (2016). |
| | | | | | helped protect the | | |
| | | | | | protein structure. | | |
| Staphylokinase | PEG | 5, 20 kDa | 1 chain | Conformational | PEG remains flexible, but | Yes | Mu et al. |
| | | | | | forms a hydration layer | | PLoS One |
| | | | | | around the protein which | | 8, 1-10 |
| | | | | | results in steric shielding. | | (2013). |
| Cytochrome c | PEG | 5 kDa | 80% of | Thermal | Conjugation caused | | Garcia- |
| | | | amino | and | thermodynamic | | Arellano et al. |
| | | | groups | Conformational | destabilization, but | | Bioconjug. |
| | | | | | polymers energetically | | Chem. |
| | | | | | trapped the destabilized | | 13, 1336-44 |
| | | | | | protein conformation | | (2002). |
| Insulin | PEG | 10, 50, | | Conformational | PEG-protein interactions | Yes | Yang et al. |
| | | 100, 200 | | | are driven by | | Biochemistry |
| | | ethylene | | | hydrophobic interactions | | 50, 2585-93 |
| | | oxide units | | | causing water to be | | (2011). |
| | | | | | excluded from the | | |
| | | | | | protein surface to | | |
| | | | | | increase structural | | |
| | | | | | stability. | | |
| WW domain | PEG | 1-45 | 1 chain | Conformational | PEG disrupts the solvent- | Yes | Pandey et al. |
| of human | | ethylene | | | shell structure and water | | Bioconjug. |
| protein | | oxide units | | | is released into the bulk. | | Chem. |
| Pin 1 | | | | | Stability is dependent on | | 24, 796-802 |
| | | | | | polymer attachment site | | (2013). |
| | | | | | and molecular weight. | | |
| | | | | | PEG provides stability by | | |
| | | | | | favorable interactions | | |
| | | | | | with protein surface | | |
| | | | | | residues in a transition | | |
| | | | | | state. | | |
| Recombinant | PEG | 20 kDa | 1 chain | Thermal | Thermal stability of | — | Natalello et al. |
| human | | | | | conjugates was increased | | PLoS One |
| methionyl- | | | | | due to a reduction in | | 7, 1-9 |
| | | | | | propensity to aggregate | | (2012). |
| Recombinant | glycoPEG | Linear: | Linear: | Thermal | Thermal stability of | No | Plesner et al. |
| human | | 10 kDa | 3 chains | | conjugates was | | Int. J. |
| factor | | Branched: | Branched: | | increased, but was | | Pharm. |
| VIIa | | 40 kDa | 2 chains | | independent of PEG | | 406, 62-68 |
| | | | | | molecular weight. This | | (2011). |
| | | | | | occurs because PEG | | |
| | | | | | postpones thermally | | |
| | | | | | induced aggregation | | |
| | | | | | leading to irreversible | | |
| | | | | | inactivation. | | |

Poly(ethylene glycol) (PEG),
poly(carboxybetaine acrylamide) (pCBAm),
poly(quarternary ammonium methacrylate) (pQA),
poly (sulfonate methacrylate) (pSMA),
poly(oligoethylene glycol methacrylate) (pOEGMA),
semi-telechelic poly[N-(2-hydroxypropyl)methacrylamide (ST-HPMA),
acrylamide (Am),
dimethyl acrylamide (DMAm),
oligo(ethylene oxide) methyl ether acrylate (OEOA),
phosphoroylcholine methacrylate (PCMA),
acrylic acid (AA),
dimethylaminoethoxy methacrylate (DMAEMA),
N-acryloyl-D-glucosamine (AGA),
poly(N-isopropylacrylamide) (pNIPAAm)

Since the majority of studied conjugates use PEG as the polymer and heat as the source of destabilization, electrostatic interactions are often overlooked. Electrostatic interactions become an important factor, however, when studying charged polymers in conditions where both the polymer and protein are changing protonation states (acidic or basic environments). Acid and base stabilization of protein-polymer conjugates is relevant for both medicinal and industrial applications. For example, therapeutic conjugates delivered orally would need to remain active in the acidic environment of the stomach (pH 1) and xylanase conjugates would need to be active in alkaline conditions during the bleaching step in the pulp and paper industry. In these environments, polymers could then either be attracted to or repelled from the protein surface based on charge which, as theorized above, could influence its stability. In terms of activity, charged polymers could also alter a proteins ability to bind a specific substrate. Thus, a library of 15 protein-polymer conjugates was created using α-chymotrypsin as a model protein and “grafting-from” ATRP techniques to grow polymers of varying charge (zwitterionic, positive, negative, and neutral), hydrophobicity, and three molecular weights for each polymer type. Michaelis-Menten kinetics were measured over a range of pH (4-10) to determine the polymer's impact on activity. Stability against acid (pH 1) and base (pH 12) were also determined and correlated with tertiary structure changes using tryptophan fluorescence. Performing the stability assays with conjugates of different physicochemical properties at the extremes of pH, where pKₐ values of both polymers and protein residues were crossed, would allow for a determination as to whether stabilization was solely due to molecular weight, electrostatic interactions, or hydrophobic (VDWs) interactions. These experiments would allow for an all-encompassing mechanistic explanation for the driving force behind protein-polymer conjugate activity and stability with pH.

In order to fully harness the potential of protein-polymer conjugates, systematic and comprehensive studies were performed to determine the polymer's physicochemical properties effects on enzymatic activity and stability. “Grafted-from” conjugates were prepared via ATRP with polymers of varying charges and pKas (zwitterionic, neutral, positive, negative) using α-chymotrypsin as a model protein. For each of these polymer types, three conjugates with increasing polymer chain length were also prepared. In these experiments, Michaelis-Menten activity was examined over a range of pH (4-10), stability against acid (pH 1) and base (pH 12) was studied, and correlated changes in enzymatic function to structural change were used to deduce structure-function relationships of protein-polymer conjugates. A mechanistic understanding of these relationships will help guide the rational design of future conjugates with maintained or enhanced function.

### Results

### Conjugate Synthesis and Characterization

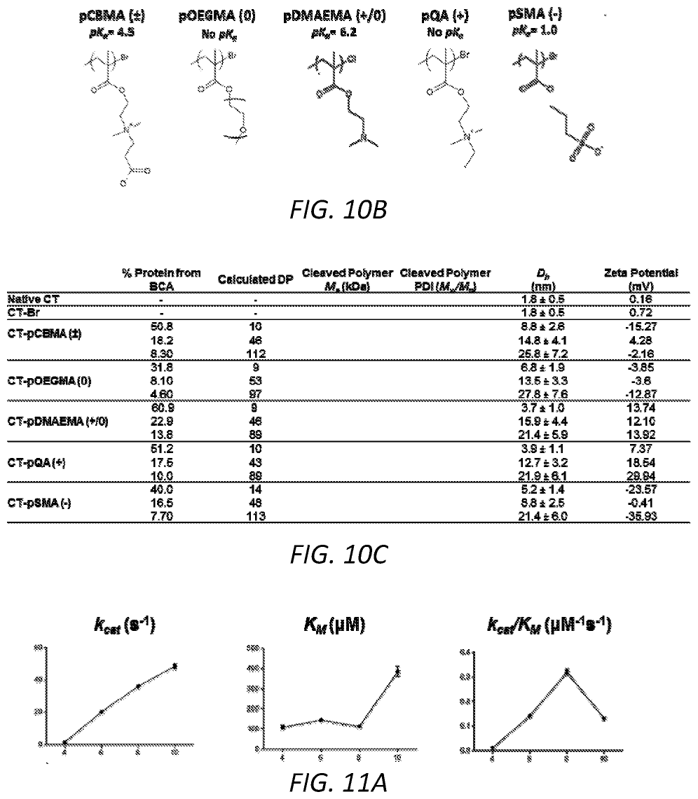

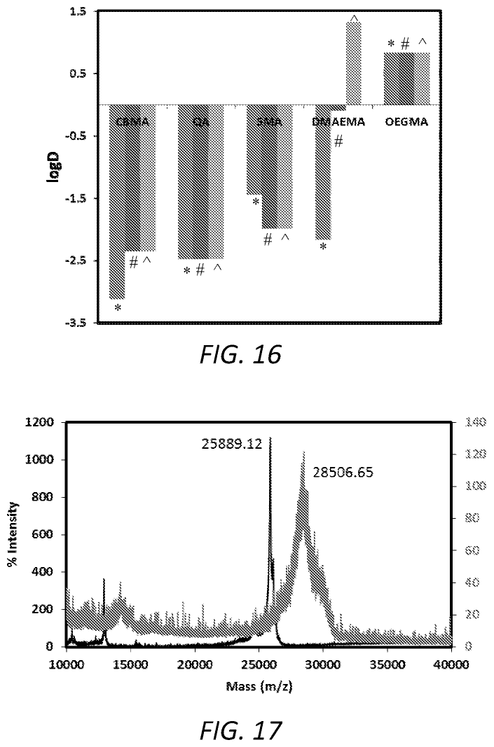

Protein-polymer conjugates were synthesized with varying polymer charge, hydrophobicity (FIG. 16), and chain length in order to determine whether electrostatic or van der Waals (VDWs) were the driving force for altered protein function or if it was simply due to polymer chain length for a densely modified protein. The synthesis scheme is shown in FIG. 10A. First, surface accessible primary amine groups were modified with an ATRP initiator (NHS-Br) as previously described (Murata et al. (2013)) which resulted in 12 modified sites through matrix assisted laser desorption/ionization time-of flight mass spectroscopy (MALDI-ToF MS) analysis (FIG. 17) (Carmali et al. (2017)). Next, zwitterionic poly(carboxybetaine methacrylate) (pCBMA ±), neutral poly(oligoethylene glycol methacrylate) (pOEGMA), neutral to positive poly(dimethylaminoethyl methacrylate) (pDMAEMA +/0), positive poly(quarternary ammonium methacrylate) (pQA +), or negative poly(sulfonate methacrylate) (pSMA −) were grown from the surface of CTBr using ATRP (FIG. 10B and Table 18). For each polymer type, three conjugates of increasing chain length, or degree of polymerization (DP), were synthesized: short (DP˜10), medium (DP˜50), and long (DP˜100). After purification, conjugates were characterized with a bicinchoninic acid (BCA) assay for protein content and DP estimation, polymer cleavage followed by gel permeation chromatography for polymer molecular weight and dispersity, dynamic light scattering (DLS) for hydrodynamic diameter, and zeta potential for interfacial electric potential (FIG. 10C). As DP increased for each conjugate type, the molecular weight increased while maintaining low dispersities, hydrodynamic diameters increased, all conjugates had increased diameters over native CT (1.8±0.5 nm), and zeta potential values were as expected from the polymer charge. Overall, 15 conjugates were prepared to create a library of samples with varying charge, hydrophobicity, and chain length.

Conjugate Activity with pH

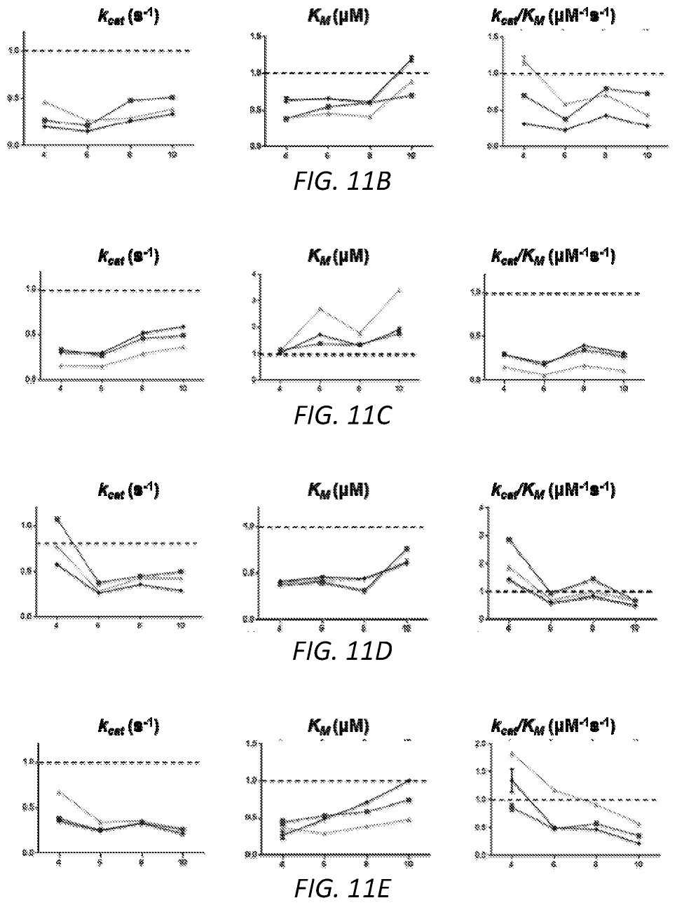

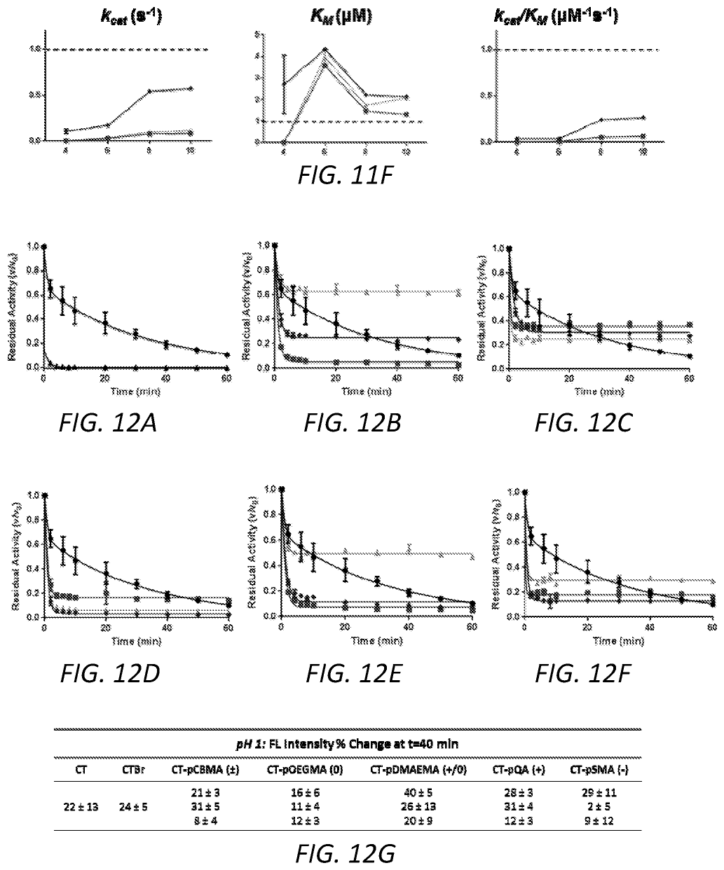

Polymer conjugation to enzymes is known to alter activity and shift the pH range in which the enzyme remains active. It was hypothesized that activity was impacted by polymer charge rather than chain length. Conjugate Michaelis-Menten kinetics were determined as a function of pH (4, 6, 8, 10) and compared to native CT (FIG. 11A) at 37° C. A hydrophobic, negatively charged substrate, N-succinyl-L-Ala-L-Ala-L-Pro-L-Phe-p-nitroanilide (suc-AAPF-pNA), was used for enzyme hydrolysis. When normalized to native CT at each pH (FIGS. 11B-11F), all conjugates showed decreased turnover rate (k_{cat}) values which has been attributed to structural stiffening (see Rodriguez-Martinez et al. Biotechnol. Bioeng. 101, 1142-9 (2008)). Substrate affinity (K_{M}) values were dependent on polymer type and lower K_{M} values indicate improved substrate binding. CT-pCBMA(±) (FIG. 11B) had lower K_{M} values compared to native CT at pH 4, 6, 8 and increased closer to native CT at pH 10. Lower K_{M} values are hypothesized to be caused by pCBMA's super-hydrophilicity (Cao and Jiang Nano Today 7, 404-413 (2012); Keefe and Jiang Nat. Chem. 4, 59-63 (2011)). Water molecules are drawn away from the active site increasing the hydrophobic interaction between the active site and substrate. Overall activity (k_{cat}/K_{M}) had decreased values compared to native CT. In contrast, neutral CT-pOEGMA (FIG. 11C) showed similar decreases in k_{cat} compared to CT-pCBMA, but showed increased K_{M} values compared to native CT leading to lower activities (k_{cat}/K_{M}). Increased K_{M} values for CT-pOEGMA are hypothesized to be caused by pOEGMA's amphiphilicity which reduces the hydrophobic-hydrophobic driving force of the substrate binding to the active site (Cao and Jiang Nano Today 7, 404-413 (2012)). CT-pDMAEMA (FIG. 11D) showed similar decreases in k_{cat} values as the other conjugate types, but increased at low pH. pDMAEMA has a pKₐ around 6.2 and will be positively charged at pH below this value. The addition of positive charge has been shown to increase enzyme activity at low pH because it reduces the pKₐ of the His 57 in the active site, which needs to be neutral to participate in the catalytic triad (Thomas et al. Nature 318, 375-376 (1985)). K_{M} values for CT-pDMAEMA were less than native CT indicating stronger substrate binding due to its hydrophilicity and favorable electrostatic interactions. CT-pQA (+) (FIG. 11E) showed similar trends to CT-pDMAEMA with increased activities at lower pH. K_{M} values were decreased in comparison to native CT indicating stronger substrate binding due to favorable electrostatic interactions between the negatively charged substrate and positively charged pQA by increasing the local concentration of substrate around the active site. Finally, CT-pSMA(−) (FIG. 11F) had decreased k_{cat} values in comparison to native CT, but k_{cat} increased at higher pH 8-10. In fact, activity was completely lost at pH 4 and 6 and was only measurable for the shortest CT-pSMA conjugate. K_{M} values were increased over native CT indicating poor substrate binding due to unfavorable electrostatic interactions between the negatively charged substrate and negatively charged pSMA by decreasing the local substrate concentration around the active site.

Overall, CT-pCBMA(±), CT-pDMAEMA(+/0), and CT-pQA(+), maintained the most activity while CT-pOEGMA(0) and CT-pSMA(−) had the least activity. Polymer length does not have a significant effect on overall activity. Also, all conjugate types had similar decreases in k_{cat} from native CT. Thus, the cause for changes in observed activity between different charged conjugates came mainly from changes in K_{M}, which is tailorable for a desired substrate.

### Conjugate Stability Against Acid

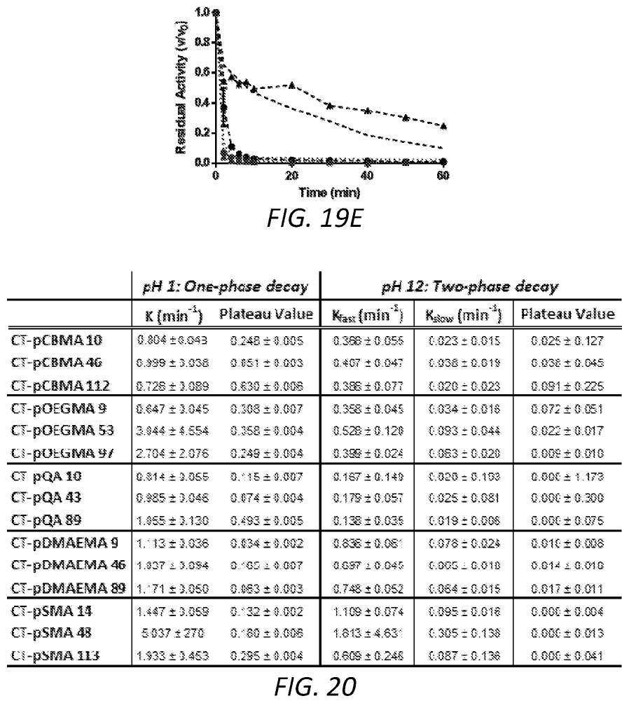

To determine how the polymer either stabilized or destabilized the conjugates when placed in a denaturing environment, such as pH 1 acid, where the protonation states of both the protein and polymer will be changed, residual activity experiments were performed. Briefly, conjugates were incubated in pH 1 acid (167 mM hydrogen chloride) at 37° C. and aliquots were taken out at specified times over 60 minutes and the residual activity was measured at pH 8 and compared to native CT and CTBr (FIGS. 12A-12F). Comparing the residual activity of native CT to CTBr (FIG. 12A), CTBr significantly lost stability within the first 2 minutes. Protein stability is often described in terms of hydrophobic collapse, and the balance of surface charge is considered less important because the residues hydrogen-bonding with the solvent will be the same in the folded and unfolded states. However, it has been shown that surface charge is, in fact, an important factor because it helps maintain optimal electrostatic interactions between surface residues that hold the protein structure together (Strickler et al. Biochemistry 45, 2761-2766 (2006)). Interestingly, the stability lost after initiator modification was regained and even enhanced after polymer growth, but was dependent on both polymer type and chain length. CT-pCBMA (±) (FIG. 12B) and CT-pQA (+) (FIG. 12E) displayed significantly enhanced stabilities and the longest polymer conjugates were more stabilizing than the shorter ones. The longest CT-pCBMA (±) maintained ˜65% of its activity while the longest CT-pQA (+) maintained ˜55% of its activity after 60 minutes. CT-pOEGMA (0) and CT-pDMAEMA (+/0) and CT-pSMA (−) did not display a length dependence on stability (FIGS. 12C, 12D, and 12F). We previously hypothesized that conjugate stability was due to either polymer preferential binding to or exclusion from the protein surface driven by electrostatic and/or hydrophobic interactions (Cummings et al. (2017)). In order to differentiate these two driving forces, residual activity measurements were also performed while independently doping in either 1.0 M NaCl or 10 v/v % dimethyl sulfoxide (DMSO) during incubation at pH 1 to disrupt electrostatic and hydrophobic interactions, respectively, between the polymer and protein surface (FIGS. 19A-19E). Stability decreased for all conjugates with the addition of NaCl and DMSO indicating that there is alternative mechanism at play. It is also worth highlighting the unique shape of the residual activity profiles for the conjugates (one-phase decay) compared to native CT (two-phase decay). Independent of polymer type or length, all conjugate activity was lost, with varying rates (FIG. 20), within the first 5 minutes of incubation at pH 1. The residual activity after this point remained constant. This suggested that the conjugated polymers were interacting with the enzyme in a specific fashion.

The three-dimensional structure of a protein is important for its function. In order to correlate changes in stability with changes in enzyme tertiary structure, tryptophan fluorescence after 40 minutes incubation at pH 1 was also measured. At this time point, the conjugates were diluted back into pH 8 buffer (similar to residual activity measurements), and the fluorescence intensity percent changes from their time 0 (no incubation at pH 1) point were determined. Comparing the residual activity to changes in tryptophan fluorescence (FL) intensity (FIG. 12G), the conjugates that were able to maintain the most activity after incubation at pH 1 also had the lowest percent change in FL intensity, implying that these conjugates were able to either maintain or refold back to their native form most effectively. This is the case for CT-pCBMA (±) and CT-pQA (+) long conjugates. Conversely, the FL intensity % change for CT-pOEGMA (0) and CT-pDMAEMA (+/0) followed similar trends to the residual activity experiments.

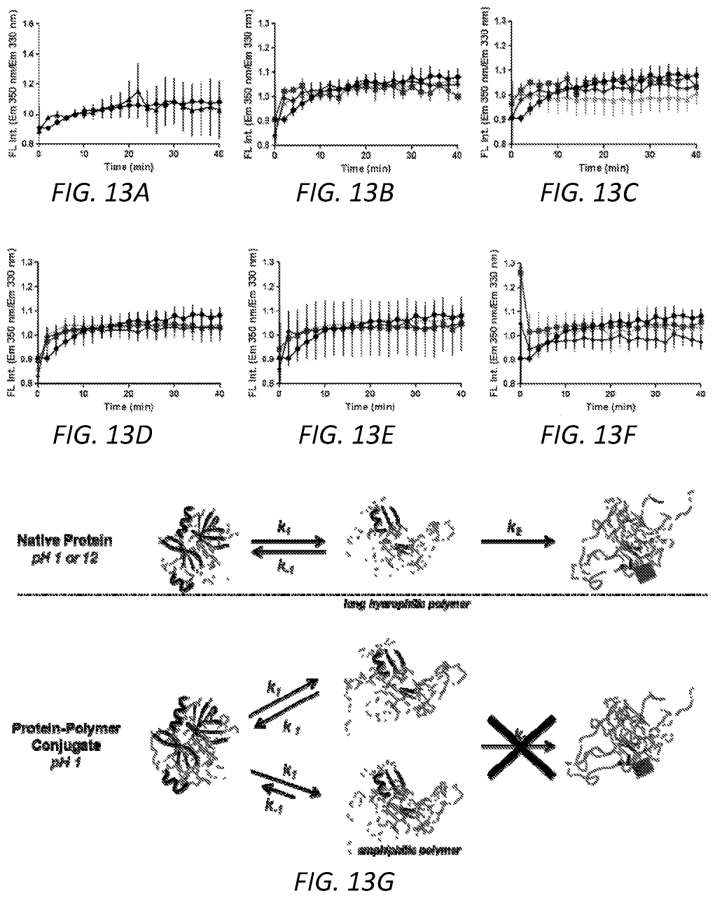

To further investigate conjugate dynamics, tryptophan FL intensity was monitored kinetically during incubation at pH 1 over 40 min (FIGS. 13A-13F). Surprisingly, the shapes of the curves were similar to the residual activity curves, where there was an immediate increase in FL intensity indicating unfolding, followed by relatively stable measurements. Interestingly, there was no length dependence for CT-pCBMA (±) and CT-pQA (+), which was an observable trait in the residual activity curves. In fact, all conjugates unfolded in a similar manner, except for CT-pSMA (−). CT-pSMA (−) at time 0 minutes is already unfolded, but becomes more folded when placed in pH 1 acid. Previous experiments indicated that surface charge was important in maintaining stability when comparing CT with CTBr. The data for CT-pSMA (−) further corroborates this finding. Replacing the previously positive primary amines with a neutral initiator, then adding negative charges with pSMA (−), causes electrostatic repulsion between charged surface residues promoting the CT-pSMA (−) conjugate to lose its tertiary structure. This can also help explain why CT-pSMA (−) had the lowest overall catalytic efficiency (k_{cat}/K_{M}) of all conjugates. This effect is reduced at pH 1, however, because the polymer will be protonated (pKₐ˜1, verified by an increase in zeta potential at pH 1=−0.52 mV for long CT-pSMA) which will decrease the electrostatic repulsion and allow the enzyme to fold back into its proper tertiary structure. Combining the residual activity, tryptophan FL refolding percent changes, and tryptophan FL unfolding over time, all conjugates appeared to unfold similarly at pH 1 independent of charge and chain length, however, certain polymers aided in the refolding step when placed back at pH 8. This phenomenon is only true, however, for the longer hydrophilic polymers (CT-pCBMA (±) and CT-pQA (+)). Previous reports on conjugate stability focused on the polymer's interaction with the protein surface; however, these experiments show the conjugated polymer's interaction with a partially unfolded protein is crucial. Langevin dynamics simulations have shown that, in the presence of free polymer, the kinetics of protein folding follow a two-step mechanism that varies with polymer hydrophobicity (Lu et al. J. Phys. Chem. B 111, 12303-12309 (2007)). In another study, Monte Carlo simulations investigated tethered polymers on a surface around a binding site and found that longer polymers forced each other into an upright position due to mutual crowding, similar to the formation of polymer brushes (see Rubenstein et al. Phys. Rev. Lett. 2012, 108, 208104). The length of the polymers also had a non-monotonic effect on the brush and depended on grafting density. Based on these studies, a proposed mechanism for conjugate acid stability is provided at FIG. 13G.

Native protein reversibly unfolds to an intermediate state and further proceeds to irreversibly unfold to a denatured state as a two-phase decay. Conjugates reversibly unfold to intermediate states, but do not proceed to fully denatured states, which are observed as one-phase decays. Therefore, polymers stabilize the intermediate form and aid in the refolding step, which is kinetically dependent on polymer hydrophobicity. More hydrophobic/amphiphilic polymers (pOEGMA (0) and pDMAEMA (+/0)) favorably bind to the aromatic, hydrophobic residues in the protein core once they are exposed. This helps stabilize the intermediate form, but hinders the protein from refolding back to its native conformation. Conversely, more hydrophilic polymers (pCBMA (±) and pQA (+)) both stabilize the intermediate form and promote more efficient refolding since there is less interaction between the hydrophilic polymer and hydrophobic protein core. Also, the potential degree of interaction decreases as chain length increases past a critical length where the polymers are able to interact with each other to form a polymer brush (for a constant grafting density). This effect is more prominent for hydrophilic polymers since amphiphilic polymers (pOEGMA (0) and pDMAEMA (+/0)) will bind favorably to exposed aromatic residues even at the longest length, which explains why there is no length dependence in the residual activity experiments for these conjugates. Finally, CT-pSMA (−) is already partially unfolded at pH 8 caused by an imbalance of charges on the protein surface. Due to pSMA's hydrophilicity, it is able to refold at pH 1, but unfolds again when placed back in pH 8 buffer causing further loss in residual activity. Overall in acid, conjugated polymers stabilize intermediate states, prevent denaturation to fully unfolded states, and aid in refolding dependent on polymer hydrophobicity and chain length. Stabilizing polymers are hydrophilic and long enough to form a brush around the protein surface thereby minimizing interactions with the partially unfolded protein core.

### Conjugate Stability Against Base

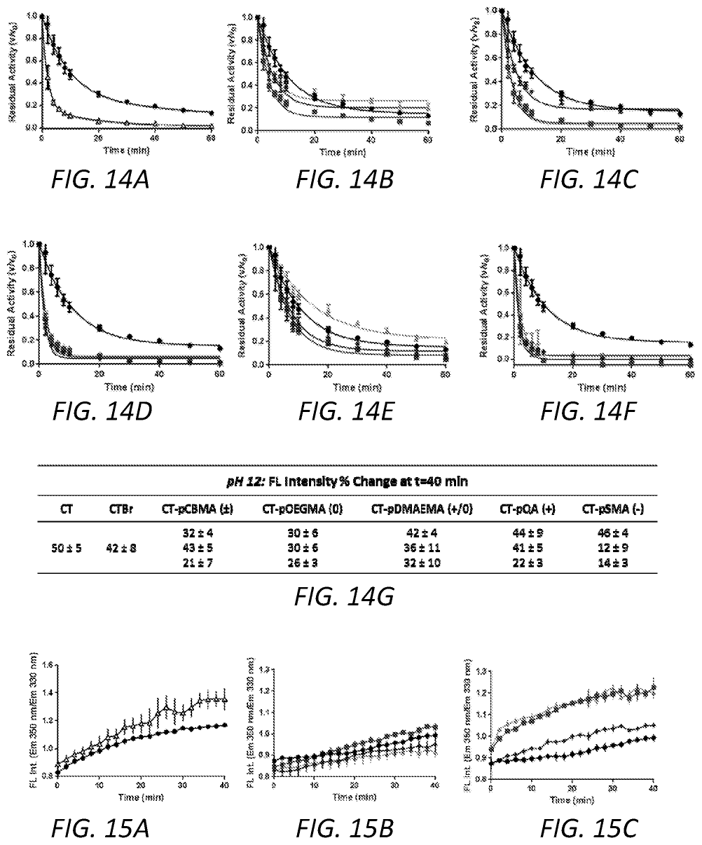

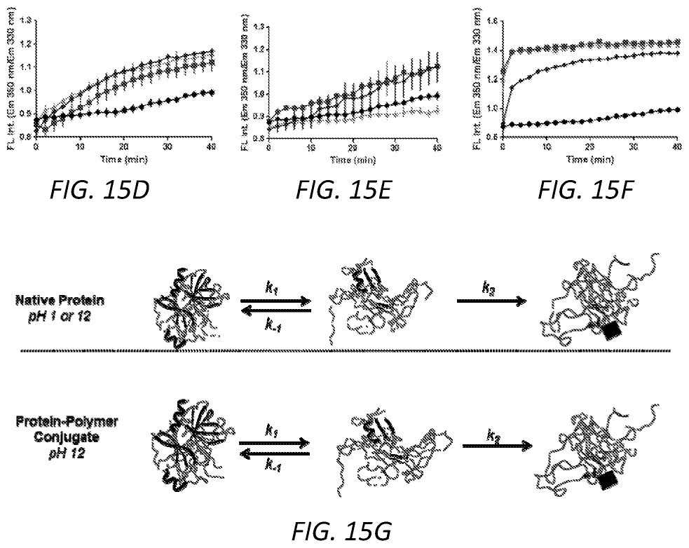

To determine whether the mechanism for conjugate stability in acid was similar to conjugate stability in base, where most residues will be deprotonated, similar residual activity were performed refolding tryptophan FL at 40 min, and tryptophan FL over time when placed in pH 12 (10 mM sodium hydroxide) solution. Comparing residual activity of native CT and CTBr, changes in the surface charge were observed to cause a loss in stability (FIG. 14A). In contrast to acid stability, however, stability against base was surprisingly not regained upon conjugating polymer (FIGS. 14B-14F). All conjugates follow a two-phase decay similar to native CT (FIG. 20). Analysis of the tryptophan FL percent change after 40 minutes incubation at pH 12 showed that conjugates were not able to fold back to their native conformation and this was independent of polymer charge and chain length. Next, unfolding was monitored kinetically using tryptophan FL intensity over 40 minutes at pH 12 (FIGS. 15A-15F). This data showed that protein folding occurred slowly over time, that polymer did not prevent complete denaturation, and that there was no dependence on polymer length. Unfolding profiles are similar for conjugates with polymers of similar hydrophobicities (pCBMA (±) and pQA (+) versus pOEGMA (0) and pDMAEMA (+/0). It was interesting to note the rapid unfolding of CT-pSMA (−) at pH 12 where both the polymer and protein residues will be deprotonated and net negatively charged further increasing electrostatic repulsion to promote unfolding.

In a general sense, protein unfolding caused by pH is due to electrostatic effects as protonation states are changing. Previous studies of myoglobin unfolding in an alkaline environment found that there was no intermediate state in the unfolding process which was attributed to the dissociation of the heme group (Sogbein et al. J. Am. Soc. Mass Spectrom. 11, 312-319 (2000)). Another study investigated the unfolding of barstar protein at pH 12 and hypothesized that deprotonation of tyrosine (pKₐ=10.5), lysine (pKₐ=10.8), and arginine (pKₐ=12.5) residues caused mutual charge repulsion causing destabilization and simultaneous loss in tertiary and secondary structure (Rami and Udgaonkar Biochemistry 40, 15267-79 (2001)). This effect was further evidenced in the unfolding of lectin at high pH (Khan IUBMB Life 59, 34-43 (2007)). It is also known that hydrogen-bonding that holds secondary structures together, such as alpha helices and beta sheets, depends on pH (Wood Biochem. J. 143, 775-777 (1974)).

The mechanism of conjugate base stability is hereby proposed to be a two-step unfolding pathway where the partial unfolding to an intermediate step is not stabilized by conjugated polymer and irreversible unfolding continues. Deprotonation of exposed tyrosine residues (pKₐ=10.5) in the intermediate state at pH 12 further disrupts the integrity of the protein hydrophobic core. Simultaneously, the hydrogen bonds forming the protein secondary structure can break and those hydrogen ions can associate with the surplus of hydroxide ions at pH 12. The combined disruption of charge in the protein's interior with the increased potential loss in secondary structure causes irreversible unfolding as the second step in the pathway which is not prevented by conjugated polymer.

### Discussion

A fundamental understanding of the underlying mechanisms for protein-polymer conjugate activity and stability in non-native environments should be understood before scientists and engineers can fully exploit their potential use. In doing so, optimized conjugates can be designed a priori for a specific application. Here the structure-function relationships of protein-polymer conjugates was explored and determination was made regarding how a conjugated polymer's interaction with the protein affects its function depending on the polymer's physicochemical properties (charge, hydrophobicity, and chain length). First, Michaelis-Menten enzymatic activity of CT-conjugates at different pH (4, 6, 8, 10) were determined and it was found that polymer charge, rather than chain length, was the dominant factor for altered activity. Specifically, a change in activity between differently charged conjugates was due to a change in K_{M} and not a change in k_{cat}. The data also showed that pH profiles can shift such that CT becomes more active at lower pH when positively charged polymers are conjugated. Conjugate residual activity and structural changes were also studied over time to determine the conjugates' stability in both acidic (pH 1) and basic (pH 12) environments. The balance of surface charge was important for protein stabilization as evidenced by rapid loss in activity of CTBr where previously positively charged primary amines were modified with neutral ATRP initiators. This loss in stability was reversed and enhanced upon polymer conjugation, except for CT-pSMA (−). Conjugate unfolding pathways at pH 1 and pH 12 with new mechanistic insight are also presented. At pH 1, conjugated polymers are able to stabilize intermediate states of partially unfolded protein and aid in reversible refolding while preventing irreversible unfolding. The conjugated polymer's interaction with the partially unfolded state determines its fate. More hydrophilic polymers increase stability by minimizing binding to the partially unfolded protein core which allows reversible refolding. Stabilization also increases as polymer chain length increases past a critical length where polymer-polymer interactions begin to form a “brush,” thereby decreasing interactions with the partially unfolded protein. More hydrophobic/amphiphilic polymers, on the other hand, bind to the partially unfolded protein, thus stabilizing it, but hindering the protein from folding back to its native conformation. This phenomena is not strongly dependent on polymer chain length. At pH 12, conjugated polymers do not stabilize intermediate forms and the unfolding pathway proceeds to complete unfolding. This is most likely due to deprotonation of exposed tyrosine residues and simultaneous breakage of secondary structure. Overall, these studies elucidate the underlying intermolecular interactions between proteins and polymers of protein-polymer conjugates and provides insight on how to increase their activity and stability by tuning polymer type. These new findings can be applied to the rational design of protein-polymer conjugates to further broaden their use and increase their impact in many fields.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A protein-polymer conjugate, comprising at least one polymer covalently conjugated to a protein, wherein the at least one polymer stabilizes a partially unfolded state of the conjugated protein when the conjugate is in an environment having a pH of about 3.0 or less, and wherein the conjugate is resistant to complete denaturation in the environment.

2. The conjugate of claim 1, wherein the conjugate is resistant to complete denaturation in an environment having a pH of about 1.0.

3. The conjugate of claim 1, wherein the at least one polymer comprises from about 10 monomeric units to about 200 monomeric units.

4. The conjugate of claim 1, wherein the conjugated protein is capable of refolding to a native state when the conjugate is subsequently in an environment (a) having a pH above about 3.0, or (b) having a pH of from about 5.5 to about 8.5.

5. (canceled)

6. The conjugate of claim 1, wherein the protein is selected from the group consisting of an antibody, an Fc fusion protein, an enzyme, an anti-coagulation protein, a blood factor, a bone morphogenetic protein, a growth factor, an interferon, an interleukin, a thrombolytic agent, a protein or peptide antigen, and a hormone.

7-9. (canceled)

10. The conjugate of claim 1, wherein when the conjugate is in an environment having a pH of about 3.0 or less, the conjugated protein has a half-life of at least about 125% of the half-life of the protein in its native state when exposed to an environment having a pH of about 3.0 or less.

11. The conjugate of claim 1, wherein the conjugated protein is an enzyme, and the enzyme retains at least about 50% of its enzymatic activity when the conjugate is in an environment having a pH of 3.0 or less.

12-18. (canceled)

19. The conjugate of claim 16, wherein the conjugate comprises a plurality of polymers, and wherein each polymer in the plurality of polymers comprises monomeric units of the same type.

20. The conjugate of claim 16, wherein the conjugate comprises a plurality of polymers, and wherein the plurality of polymers comprises a first polymer and a second polymer, wherein the first polymer and the second polymer are each comprised of monomeric units of a different type.

21. The conjugate of claim 1, wherein the at least one polymer comprises a positively charged polymer.

22. The conjugate of claim 21, wherein the positively-charged polymer is poly(quaternary ammonium methacrylate) (pQA).

23. (canceled)

24. The conjugate of claim 1, wherein the conjugate specifically binds to mucin.

25-45. (canceled)

46. A composition comprising the conjugate of claim 1 and a pharmaceutically acceptable excipient.

47. (canceled)

48. The composition of claim 46, wherein the composition is formulated for oral, rectal, intranasal, or intravaginal administration to a subject.

49. (canceled)

50. A method of enhancing the delivery of a protein to the intestinal tract of a subject, the method comprising administering to the subject a pharmaceutical composition comprising a protein-polymer conjugate, wherein the conjugate comprises at least one polymer covalently conjugated to a protein, wherein the at least one polymer stabilizes a partially unfolded state of the conjugated protein when the conjugate is in an environment having a pH of about 3.0 or less, and wherein the conjugate is resistant to complete denaturation in the environment.

51. The method of claim 50, wherein the conjugate is resistant to complete denaturation when exposed to an environment having a pH of about 1.0.

52. The method of claim 50, wherein when the conjugate is in an environment having a pH of about 3.0 or less, the conjugated protein has a half-life of at least about 125% of the half-life of the protein in its native state when exposed to an environment having a pH of about 3.0 or less.

53. The method of claim 50, wherein the conjugated protein is an enzyme, and the enzyme retains at least about 50% of the enzymatic activity of the native enzyme when the conjugate is in an environment having a pH of about 3.0 or less.

54-57. (canceled)

58. The method of claim 50, wherein the conjugated protein refolds to a native state when the conjugate is in an environment having a pH of from about 5.5 to about 8.5.

59. (canceled)

60. The method of claim 50, wherein the at least one polymer comprises a positively-charged polymer.

61-85. (canceled)
